(19) United States
(12) Patent Application Publication
(10) Pub. No.: US 2003/0130121 A1
(43) Pub. Date: Jul. 10, 2003

(54) Novel bacterial isolate and the preparation and use of its active metabolites

(76) Inventors: Berndt Gerhardson, Uppsala (SE); Christian Thaning, Knivsta (SE); Ragnar Weissmann, Uppsala (SE); Christopher Folkeson Welch, Uppsala (SE); Jolanta Borowicz, Uppsala (SE); Rikard Hedman, Uppsala (SE)

Correspondence Address: BACON & THOMAS, PLLC, 625 SLATERS LANE, FOURTH FLOOR, ALEXANDRIA, VA, 22314

(21) Appl. No.: 10/130,291

(22) PCT Filed: Nov. 30, 2000
(86) PCT No.: PCT/SE00/02391

(30) Foreign Application Priority Data
Nov. 30, 1999 (SE) 9904334-1
Aug. 24, 2000 (SE) 0002999-1

(51) Int. Cl. A01N 63/00; C12N 1/20

(52) U.S. Cl. 504/117; 435/252.1; 424/93.4

(57) ABSTRACT

The invention relates to a new biocontrol agent and its active metabolites for use in controlling weeds and/or fungal growth and differentiation, the biocontrol agent being a strain (A 153) of the bacterium Serratia plymuthica, an isolate of which has been deposited at NCIMB and received the accession No. 40938, and its active metabolites being haterumalide A, B, E and X and derivatives thereof. The invention also relates to weed and fungus suppressing compositions and methods for suppressing weeds and fungi using said bacterial strain or its active metabolites or said compositions.

### FIELD OF THE INVENTION

The present invention relates to a new biological control agent (biocontrol agent) and active metabolites thereof for use in controlling weeds and/or fungal growth and differentiation. More specifically, the invention relates to a novel bacterial strain, A 153 (Serratia plymythica), and the use of compositions containing this bacterial strain in order to suppress or control weeds, especially annual herbaceous weeds, in agricultural crops, but also in horticultural and silvicultural systems, without deleteriously affecting the crop, and/or fungal growth or differentiaton, and further relates to the preparation and use of active metabolites of said novel strain as biofungicides for controlling fungal diseases of plants humans and animals and as bioherbicides for controlling weeds.

### BACKGROUND OF THE INVENTION

Weeds Control

Weeds cause greater losses on agricultural land than all other pests combined, and conventional herbicides used for controlling them account for half of the global agrochemical sales. Agricultural weeds proliferate rapidly and actively, often have long taproots and compete with crops for nutrients, light, water and space; they interfere with harvesting or produce allelopathic substances, all in all results in lower yields as well as reduced qualities of the crop and food product affected.

Despite the dominant use of conventional chemical methods—contact, residual and systemic pesticides—for weed control, physical/cultural methods such as tillage, crop rotations, crop competition and cover crops remain important weed management tools in many modern systems of crop production. However, all these standard methods have drawbacks either in terms of being costly and energy-demanding, or by having adverse or undesired effects on environment, notably contaminating ground water, and on human health. Chemical pesticides are often highly effective, but since their negative side-effects and the selection for herbicide-resistant weed populations have become widely acknowledged, this has led, along with e. g. a growing interest in organic farming, to strong pressure on governments to find alternatives. The use of biological methods such as microbial weed control may be preferable to the use of other control methods and, thus, such agents have been increasingly tried. As a result there exists now a broad base of experience and a wide range of reliable methods and technology.

However, to date, microbial weed control has mainly involved laboratory culturing and subsequent field application of indigenous pathogens. Under natural conditions such pathogens frequently occur at the end of the growing season, too late to prevent yield losses. Since they do not establish and sustain themselves at adequate levels to maintain control in the long term, they are usually applied to the target weeds annually in an inundative manner, i.e. by using heavy inoculation to achieve immediate control, in much the same way as a chemical herbicide, hence they are often described as “bioherbicides”. The term “bioherbicide” is here abandoned in favour of “weed suppressive agent”, as the purpose of the agent described herein is not to infect or primarily to kill or eradicate the target weeds, but, in line with the new biodiversity concept, the tipping of the competitive balance in favour of the crop to achieve a reduction and stabilisation of the weed population at an economically favorable level. However, the use for weed control in other systems is not excluded, for example, when a reduced vegetation in general is desired.

Furthermore, to be effective and usable as a commercial weed control product, weed suppressive agents have to be stable, give acceptable and reproducible control in the field, produce no deleterious effects on the crop plant or plants, i.e. be selective, and there must be established methods for field applications. To date no non-infecting bacterial agents have fulfilled this requirement and, thus, have been used as commercial products.

Fungal Growth Control

Fungi cause great losses in crop and forest production as well as during storage of food and feed. They also are deleterious by affecting our construction materials, e. g. in houses, and, furthermore, cause increasing problems within veterinary medicine and medicine. Such unwanted fungal growth is currently to a large extent controlled by the application of fungicides and preservatives, many of which have unwanted side effects. Resistance of fungi to these chemicals is also increasing. An environmentally sound, alternative approach to this use of fungicides is to better utilize the microbial antagonism that is already present in nature. Several types of antagonistic interactions are known and have been used in practice. Mechanisms of antagonism include competition for. nutrients or space, contact inhibition by hyphal interference, production of antibiotic substances and hyper-parasitism. Some of these antifungal mechanisms are easy to envision for replacing fungicides that are used, e. g. to protect plants against pathogens and products against spoilage molds.

Searching for novel and antifungal antibiotics commercially useful both in medicine and to control plant pathogens has been ongoing for the last fifty years and has recently gained special attention. Emergence of pathogens resistant to available antibiotics and fungicides, health concerns and new regulations about use of certain chemicals, such as e.g. methyl bromide, became a driving force in finding and developing alternatives to control emerging fungal infections. In the context of all these concerns, the vast array of antimicrobial metabolites produced by diverse and often not-well studied soil and plant-associated microorganisms, remains a reservoir of new and potentially safer antifungal compounds. The use of microorganisms for the control of fungal diseases depending of the area of interest, falls basically into three categories: use of microorganisms directly as a biological control agents (plant diseases and storage diseases); use of metabolites produced by fermentation as biofungicides and drugs; and use of these compounds as a base for the synthesis of new chemical antifungals.

There are many well-documented cases of microorganisms and among them bacteria producing effective antifungal compounds. Most cases studied involve fluorescent pseudomonads that produce antibiotics such as phenazines (Tomashow and Weller, 1988), 2,4-diacetylphloroglucinol (Keel et al., 1992; Harrison et al., 1993; Raijmakers et al., 1997), pyrrolnitrin (Howell and Stipanovic, 1979; Lambert et al., 1987), pyoluteorin (Howell and Stipanovic, 1980; Kraus and Loper; 1995), siderophores (Teintze et al., 1981; Duijff et al., 1994) and recently discovered 2,3-deepoxy-2,3-didehydrorhizoxin (Hökeberg, 1998). In contrast, much less work have been done on other ubiquitos soil and plant-inhabiting bacteria such as Stenotrophomonas sp., Burkhulderia cepacia and Serratia spp. The lack of interest in exploiting these microorganisms might be explained by arising evidence, of their potential pathogenicity with often a broad clinical spectrum. Due to this evidence, their direct use even as biocontrol agents in agriculture might be thus questionable and thus lowers their commercial value. However, several novel antibiotics in addition to prodigiosin and pyrrolnitrin of Serratia spp. (Kalbe et al., 1996; Okamoto et al., 1998), were recently discovered to be produced by these bacterial species. Maltophilin (Jakobi et al., 1996) and xanthobaccins (Nakayama et al., 1999) of respectively S. maltophilia and Stenotrophomonas spp., xylocandins/cepacidines (Meyers et al., 1987; Lee et al., 1994) and a compound AFC-BC11 (Kang et al., 1998) of B. cepacia as well as an antibiotic CB-25-I (Shoji et al, 1989) and oocydin A (Strobel et al., 1999) of respectively S. plymuthica and S. marcescens are here the good examples. The oocidin A under the name haterumalide A was almost at the same time isolated from the sponge of Ircinia sp. by Takada et al., (1999) and the structures of other forms of haterumalide were there elucidated.

There is a broad range of important fungal pathogens and among them Aspergillus fumigatus, Candida albicans, Fusarium culmorum, and Sclerotinia sclerotiorum.

The fungal, soil-borne plant pathogen Sclerotinia sclerotiorum (Lib.) de Bary has a worldwide distribution, and is an economically important pathogen in bean, soybean, sunflower and oil seed brassicae crops. Its wide host range in combination with its persistent resting structures, the sclerotia, also makes it difficult to control. Besides resistance breeding, which so far has not been very successful, the main control strategies currently used include cultural practices and/or fungicidal applications (Steadman, 1979; Kharbanda and Tewari, 1996). Fungicidal treatment is still regarded as the main, most reliable and efficient control method under most field-cropping conditions (Dueck et al., 1983).

Another strategy for controlling S. sclerotiorum is biological control (Adams, 1990), and in searching for biocontrol agents, the most intensively studied organisms are a number of mycoparasitic fungi that are able to parasitize and kill the sclerotia in the soil (Whipps and Budge, 1990). Of these, the most thoroughly investigated is Coniothyrium minitans Campbell e.g., (Turner and Tribe, 1975.; Turner and Tribe, 1976; Trutmann et al., 1982; McQuilken et al., 1995; Gerlagh et al., 1999). Other microbes antagonistic to mycelial growth (Kalbe et al., 1996) and/or the germination of the ascospores are also reported (McLoughlin et al., 1991; Yuen et al., 1991; Pozdnyakov, 1994), but attempts to utilize these for field-scale biological control purposes are more scarce.

Yet another strategy for control in terms of interfering with the S. sclerotiorum life cycle is the utilization of agents that suppress the carpogenic germination and/or the normal development of stipes and apothecia from the sclerotia (Mc Lean, 1958; Steadman and Nickerson, 1975). Such apothecial suppression will decrease the production of ascospores, which in certain crops are the most important infection units, such as in oil-seed rape, bean and soybean. Other than fungi, organisms and compounds that may give such effects have, however, so far been little studied, and this seems to be especially the case for bacteria that affect the carpogenic germination or apothecial formation. One of the few papers, and to the best of our knowledge the only, on bacteria suppressing apothecial development, reports on different strains of Bacillus spp. that reduced apothecial formation from sclerotia in soil (Lüth et al., 1993).

However, to be effective and usable the agents have to be stable, give reproducible results in the test systems applied and there must be possibilities to apply them under commercial conditions. To date few have fulfilled these requirements and, thus, have been used as commercial products.

### BRIEF DESCRIPTION OF THE INVENTION

The present invention provides a biocontrol agent useful and effective for controlling weeds in commercial crop production and/or deleterious fungal growth. A novel strain (A 153) of the bacterium Serratia plymuthica showing the desired characteristics is provided. The isolate was deposited on the 30^{th} of March 1998 at the National Collection of Industrial and Marine Bacteria Limited (NCIMB), Aberdeen, Scotland under the terms of the Budapest Treaty and has received NCIMB Accession No. 40938.The Applicant has requested that a sample of the deposition may be submitted only to an expert determined to be entitled thereto by international or national regulations.

It has also surprisingly been found that the bacterial isolate Serratia plymuthica A 153 (NCIMB 40938) produces active metabolites having fungus growth and weeds controlling properties and that these metabolites thus can be used as biofungicides for controlling fungal diseases of plants, humans and animals and as bioherbicides for controlling weeds. Said metabolites are the macrocyclic lactones haterumalide A, B and E, which are substances known per se, and haterumalide X which is a substance not previously known.

Thus, the invention also provides the novel metabolite haterumalide X as such and the use of haterumalide A, B, E and X and derivatives thereof as biofungicides for controlling fungal diseases of plants, humans and animals and as bioherbicides for controlling weeds. The active metabolites are particularly useful for inhibiting apothecial formation and germination of ascospores as well as hyphal growth of the pathogenic fungus Sclerotinia sclerotiorum and thus can be used for inhibition of mycelial and hyphal growth of S. sclerotiorum.

The invention also provides a weeds and/or fungus growth controlling composition comprising, as active ingredient, a biocontrol agent being the above novel strain A153 of the bacterium Serratia plymuthica or a biologically pure culture thereof, or a culture broth thereof. The invention also provides compositions for controlling plant diseases caused by pathogenic fungi, particularly sclerotia-producing fungi, for controlling diseases of humans and animals caused by pathogenic fungi and for controlling weeds, said compositions comprising haterumalide A, B, E and/or X and/or derivatives thereof as active angifungal or weed-controlling ingredient. In particular, the invention provides a S. sclerotiorum-controlling composition comprising, as active ingredient, haterumalide A, B, E and/or X and/or a derivative thereof.

Further, the invention provides methods for controlling weeds and fungi based upon the ability of the novel strain A 153 and its active metabolites to suppress or regulate the weed growth and development and/or mycelial, apothecial and/or spore production in fungi. The weed and/or fungal growth controlling compositions of the invention can be applied to seeds, seedlings, plant vegetative propagation units, plant or plant parts or soil or growth substrate or other places where weeds or fungus growth control is desired.

### BRIEF DESCRIPTIONS OF THE DRAWINGS

FIG. 1 shows the UV spectrum of haterumalide A.

FIG. 2 shows the inhibition of S. sclerotiorum mycelial growth by haterumalide A applied onto antimicrobial susceptibility test discs in concentration range from 0.05 to 5 μg ml⁻¹ per disc.

FIG. 3 shows the inhibited apothecial formation on sclerotia treated with broth culture of the isolate A 153. The bar's length equals 1 cm.

FIG. 4 shows treatment effects on clover undersown in a barley field, shown as fresh weight and number of plants per square meter.

FIG. 5 shows treatment effects in Thlaspi arvense (THLAR) and Stellaria media (STEMA) in two separate field microplot experiments, expressed as percentage reduction in fresh weight compared to untreated control plots.

FIG. 6 shows the apothecia-suppressing effects in S. sclerotiorum for different concentrations of A153 broth culture. Each dot represents the mean number of apothecia developed for a given bacterial cell concentration per cm². For each concentration, N=4 (Petri dishes with 10 sclerotia per dish). The position of the black square on the y axis gives the value for the water control. Vertical bars indicate standard errors for means.

### DETAILED DESCRIPTION OF THE INVENTION

The present invention, in one aspect, relates to the use of the bacterial isolate Serratia plymuthica A 153 and the chlorinated macrolides haterumalide A, B, E and X, produced bv said bacterial isolate, and/or derivatives of said lactones as biocontrol agent and biofungicides, respectively, for controlling diseases of plants, humans and animals caused by pathogenic fungi and as bioherbicides for controlling weeds.

The bacterial Serratia plymuthica isolate A 153 was deposited on Mar. 30, 1998 at the National Collection of Industrial and Marine Bacteria Limited (NCIMB), Aberdeen, Scotland under the terms of the Budapest Treaty and has received NCIMB Accession No. 40938.

Characterisation of the Novel Bacterial Strain A 153

Morphological characteristics: Colony morphology on TSA 10 is round, white, with moderately convex colonies, and a distinct border. It is a Gram negative rod.

Biochemical characteristics: The analysis was performed using the API 20 E Rapid Identification System (bioMerieux, France). According to this test, A 153 is most similar to Serratia plymuthica, with a matching index of 98,6%.

| | Reaction of isolate A 153 |
|---|---|
| Characteristics tested | in the API 20 E rapid test |
| Beta-galactosidase | + |
| Arginine dihydrolase | − |
| Lysine decarboxylase | − |
| Ornithine decarboxylase | − |
| Citrate utilisation | + |
| Hydrogen sulfide production | − |
| Urease | − |
| Tryptophane deaminase | − |
| Indole production | − |
| Acetoin production | + |
| Gelatinase | + |
| Glucose fermentation/oxidation | + |
| Mannitol fermentation/oxidation | + |
| Inositol fermentation/oxidation | + |
| Sorbitol fermentation/oxidation | + |
| Rhamnose fermentation/oxidation | − |
| Sucrose fermentation/oxidation | + |
| Melibiose fermentation/oxidation | + |
| Amygdalin fermentation/oxidation | + |
| Arabinose fermentation/oxidation | + |
| Cytochrome-oxidase | − |

Three of the above-named active metabolites of the bacterial strain A 153 have been found to be identical with the previously reported compounds haterumalide A (also known as oocydin. A), haterumalide B and haterumalide E (Strobel et al., 1999; Ueda and Hu, 1999; Takada et al., 1999).

Haterumalide A has the following structural formula

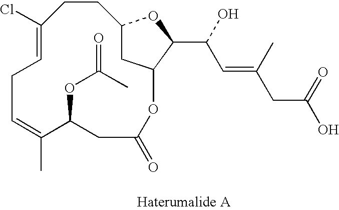

Haterumalide A is isolated as a white solid and is characterised by the following spectroscopic properties.

Optical Rotation Data

The optical rotation of compound 1 was recorded with a Perkin Elmer 241 polarimeter at 20° C.

[α]_{D}²⁰=−2.5 (c=0.50)

Mass Spectral Data

Mass spectra were recorded on a JEOL HX110 mass spectrometer. High resolution FAB spectra were recorded on a glycerol matrix. Fragmentation studies were performed in the EI mode.

| | | |
|---|---|---|
| HR-FAB-MS | Found for C₂₃H₃₁³⁵ClO₈ | 470.1750 |
| | Calculated | 470.1707 |

NMR Spectral Data

NMR spectra were recorded in CD₃OD at 26° C. with a Bruker DRX-600 spectrometer at 600 MHz for ¹H and 150 MHz for ¹³C. Chemical shifts are reported downfield from tetramethylsilan using the solvent peak as an internal reference (3.30 ppm for 1H and 49.0 ppm for ¹³C). ¹³C signals were assigned through HSQC-DEPT and HMBC correlations.

¹H NMR Spectra

¹H-NMR: δ 1.41 (ddd, 1H), 1.52 (ddd, 1H), 1.85 (s, 3H), 1.90 (s, 3H), 2.05 (s, 3H), 2.11 (br dd, 1H), 2.27 (ddd, 1H), 2.32 (dddd, app tt, 1H), 2.48 (m, 1H), 2.50 (m, 1H), 2.82 (dd, 1H), 2.85 (dd, 1H), 3.03 (d, 1H), 3.08 (d, 1H), 3.52 (ddd, 1H), 3.95 (dd, 1H), 3.97 (dddd, app tt, 1H), 4.58 (t, 1H), 5.28 (d, 1H), 5.32 (m, 1H), 5.41 (d, 1H), 5.70 (br dd, 1H), 5.81 (dd, 1H).

¹³C NMR Spectra

¹³C NMR: δ 16.9, 18.1, 20.9, 26.9, 28.1, 34.7, 38.1, 45.2, 65.5, 67.9, 75.9, 77.3, 83.6, 126.1, 130.0, 130.3, 132.5, 133.2, 134.4, 169.0, 170.6, 174.8.

Proton and carbon NMR data for haterumalide A are also evident from Table 1.

Haterumalide B has the following structural formula

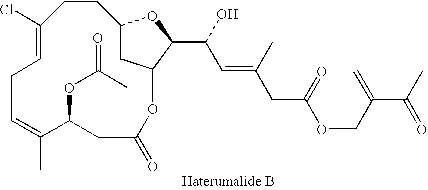

Proton and carbon NMR data for haterumalide B are evident from Table 1.

Haterumalide E has the following structural formula

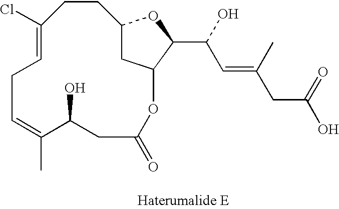

Proton and carbon NMR data for haterumalide E are evident from Table 1.

Haterumalide X is a novel compound of the following structure

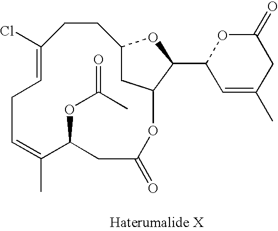

not previously known in the art and is thus claimed as such.

Proton and carbon NMR data for haterumalide X are evident from Table 1.

TABLE 1
Proton and carbon NMR data for Haterumalides A, B, E and X
obtained at 600 MHz with samples dissolved in methanol-d₄.

| | Haterumalide | | Haterumalide | | Haterumalide | | Haterumalide | |
|---|---|---|---|---|---|---|---|---|
| Posi- | A | | B | | E | | X | |
| tion^{a} | δC | δH | δC | δH | δC | δH | δC | δH |
| 1 | 169.0 | | 169.8 | | 169.0 | | 169.7 | |
| 2 | 38.1 | 2.82 | 37.5 | 2.80 | 37.2 | 2.64 | 37.9 | 2.81 |
| 2 | | 2.85 | | 2.86 | | 2.75 | | 2.85 |
| 3 | 67.9 | 5.81 | 67.6 | 5.82 | 66.0 | 4.63 | 67.4 | 5.82 |
| 4 | 134.4 | | 133.4 | | 134.3 | | 132.8 | |
| 5 | 130.3 | 5.70 | 129.8 | 5.73 | 130.0 | 5.63 | 130.1 | 5.72 |
| 6 | 26.9 | 2.50 | 26.8 | 2.53 | 27.1 | 2.29 | 27.2 | 2.47 |
| 6 | | 3.52 | | 3.52 | | 2.30 | | 3.53 |
| 7 | 126.1 | 5.28 | 125.8 | 5.33 | 125.0 | 5.29 | 126.2 | 5.33 |
| 8 | 133.5 | | 132.6 | | 133.7 | | 131.9 | |
| 9 | 34.7 | 2.27 | 34.4 | 2.30 | 34.5 | 2.29 | 34.6 | 2.30 |
| 9 | | 2.48 | | 2.48 | | 2.48 | | 2.47 |
| 10 | 28.1 | 1.41 | 28.1 | 1.42 | 27.8 | 1.38 | 27.9 | 1.41 |
| 10 | | 2.32 | | 2.32 | | 2.06 | | 2.31 |
| 11 | 77.3 | 3.97 | 77.0 | 3.97 | 76.8 | 3.91 | 77.2 | 3.97 |
| 12 | 38.1 | 1.52 | 37.8 | 1.55 | 38.1 | 1.51 | 38.0 | 1.55 |
| 12 | | 2.11 | | 2.13 | | 2.06 | | 2.13 |
| 13 | 75.9 | 5.32 | 75.5 | 5.34 | 75.7 | 5.29 | 75.6 | 5.33 |
| 14 | 83.6 | 3.95 | 83.4 | 3.93 | 84.1 | 3.88 | 83.2 | 3.93 |
| 15 | 66.5 | 4.58 | 65.7 | 4.57 | 66.4 | 4.53 | 65.3 | 4.55 |
| 16 | 130.0 | 5.41 | 130.0 | 5.43 | 130.0 | 5.37 | 127.5 | 5.34 |
| 17 | 133.2 | | 133.2 | | 133.3 | | 137.1 | |
| 18 | 45.2 | 3.03 | 44.6 | 3.14 | 46.0 | 3.05 | 49.3 | 2.93 |
| 18 | | 3.06 | | 3.15 | | 3.05 | | 2.99 |
| 19 | 174.8 | | 171.3 | | 172.3 | | 178.3 | |
| 20 | 18.1 | 1.90 | 17.5 | 1.91 | 17.4 | 1.85 | 17.4 | 1.91 |
| 21 | 16.9 | 1.85 | 16.2 | 1.84 | 16.4 | 1.83 | 16.3 | 1.85 |
| 22 | 170.6 | | 169.7 | | | | 170.1 | |
| 23 | 20.9 | 2.05 | 19.6 | 2.05 | | | 19.7 | 2.05 |
| 24 | | | 63.2 | 40.82 | | | | |
| 25 | | | 143.2 | | | | | |
| 26 | | | 199.2 | | | | | |
| 27 | | | 24.5 | 2.39 | | | | |
| 28 | | | 126.9 | 6.13 | | | | |
| 28 | | | | 6.34 | | | | |

^{a}Assignments based on COSY, HSQZ-DEPT and HMBC spectra.

The term “derivatives thereof” in connection with haterumalide A, B, E and X means, throughout the specification and claims, any compound structurally related to said haterumalides and formed, or being regarded as formed, from said haterumalides but still retaining the biological activity of said haterumalides in all essential respects. As examples of derivatives, mention can be made of functional derivatives containing one or more additional functional groups such as OH, NH₂ and the like, or with one or more of the functional groups being removed from or displaced within the compound.

Haterumalide A, B, E and X, which were identified in a bioassay guided purification procedure starting with the cell free s pernatant from the isolate of S. plymuthica followed by spectroscopic analysis, all show the ability to inhibit apothecial formation of sclerotia, ascospore germination and mycelial growth of S. sclerotiorum. The minimal inhibitory concentration to totally inhibit apothecial formation of sclerotia is 0.5 μg ml⁻¹ for all tested haterumalides. When applying both bacterial broth culture and haterumalide A, B, E and X to preconditioned sclerotia, the stipes development is curtailed and differentiation of the stipes is highly affected. Their elongation is totally inhibited, the tips become thick and melanized and yellow-brownish droplets are observed on the malformed stipes. The haterumalide metabolites of the invention being able to suppress the carpogenic germination and/or the development of stipes and apothecia from sclerotia are thus considered as potentially useful in preventing infections caused by S. sclerotiorum, the serious pathogen of crops such as oil seed rape and sunflowers.

Most of the reports on bacterial suppression of S. sclerotiorum consider the inhibition of mycelial germination of sclerotia by Bacillus spp, and Burkholderia cepacia, but not the suppression of apothecial formation. One of the reports, and to the best of our knowledge, the only one about bacterial suppression of apothecial development in soil considers also isolates of Bacillus spp. (Luth et al., 1993). We could either find reports describing Serratia spp. as a potential antagonist to S. sclerotiorum and, being of particular interest in connection with the present invention, there are no records on microbial metabolites with the ability to suppress the apothecial formation of sclerotia. Antifungal compounds found to control this fungus are pyrrolnitrin, aminopyrrolnitrin and monochloro-aminopyrrolnitrin of B. cepacia (McLoughlin et al., 1992) and two highly methylated polyketide derivatives of Penicillium spp. (Stierle et al., 1999), described however as inhibiting the mycelial growth in vitro.

Haterumalide A, B, E and X also effectively suppress, at concentration ranges from 0.4 to 15 μg ml⁻¹, germination of diverse fungal spores (Table 4, Example 8) and are especially effective when tested against ascospores of S. sclerotiorum, sporangia of Pythium spp. and, with the exception of haterumalide E, against conidiospores of F. oxysporum. At lower concentrations than these of haterumalides, pyrrolnitrin totally inhibits the conidiospore germination of F. culmorum, A. fumigtatus and M. canis although haterumalides also show the effect on these fungi. In contrast to studies by Strobel et al. (1999), which point out that oocydin A (haterumalide A) had little or no activity against a group of organisms representing the three classes of fungi other than Oomycetes, we have surprisingly found that haterumalide A, B as well as E and X have an ability to effectively inhibit spore germination of both Fungi imperfecti and Ascomycetes and, at higher concentrations, also Basidiomycetes. These diverse results might be probably explained by i: with exception of S. sclerotiorum, the use of different test fungi and ii: the use of different test methods (inhibition of mycelial growth versus inhibition of spore germination).

Although we do not wish the claimed invention to be bound to any particular theory, we are of the opinion that the mechanism by which the haterumalide metabolites of the present invention prevent both spore germination and apothecial formation involves the interference of the haterumalides with processes such as nucleic acid synthesis and microtubuline activity.

The haterumalide metabolites of the invention have also surprisingly been found to suppress or regulate weed growth and development and are therefore contemplated for use in weeds control. Thus, said haterumalides can be used as bioherbicides for suppressing or controlling weeds, especially annual herbaceous weeds, in agricultural crops without deleteriously affecting the crop.

“Control” or “controlling” in connection with the use of the haterumalides of the invention means, throughout the specification and claims, any act including killing, eradicating, suppressing and regulating the fungus or weed or the growth thereof, in accordance with the intended objective and the prevailing circumstances.

Based upon the above findings, one preferred embodiment of the first aspect of the invention relates to the use of haterumalide A, B, E and X and their derivatives for controlling soil-borne plant diseases caused by pathogenic fungi, and, in particular, for controlling Sclerotinia sclerotiorum by inhibiting mycelial and hyphal growth thereof.

Preferred commercial crops to be protected from pathogenic fungi are cereals such as barley, wheat, oats and rye.

However, haterumalide A, B, E and X and their derivatives are also contemplated for use in other commercial crops such as sugar beet, rice, maize, potato, soybean, vegetables and ornamentals.

Commercially important crops to be protected from S. sclerotiorum are bean, soybean, sunflower and oil seed brassicae crops.

Haterumalide A, B, E and X and their derivatives may also be used for controlling weeds, particularly dicotyledonous weeds. Examples of such weeds are fat hen (Chenopodium album), field pennycress (Thlaspi arvense) and chickweed (Steilaria media). Other weeds are redroot pigweed (Amaranthus retroflexus L), charlock (Sinapsis arvensis), field pansy (Viola arvensis), Lemna minor, Fallopia convolvolus, Equisetum arvense, Galeopsis spp., Polygonum spp., and Fumaria officinalis.

A further field of use of haterumalide A, B, E and X and their derivatives is the profylax and treatment of human fungal diseases, such as diseases caused by Aspergillus spp., e.g. Aspergillus fumigatus, i.e. the different forms of aspergillosis, and by Candida spp. and Fusarium spp.

Haterumalide X and derivatives thereof are also contemplated for use as cytostatic agent and, said compounds, as well as haterumahde A, B and E and derivatives thereof, are also contemplated for use as antibacterial and antiviral agents.

According to a second aspect, the invention relates to a plant fungal disease or weeds controlling composition for use according to the invention, the composition being characterized by comprising the bacterial strain A153 or haterumalide A, B, E and/or X and/or a derivative thereof in admixture with an agriculturally or horticulturally acceptable carrier or diluent. The carrier may be a liquid one or a solid porous material impregnated with the bacterial strain A153 or haterumalide A, B, E and/or X or a derivative thereof.

The composition may in addition contain other biocontrol active substances or agents.

A third aspect of the present invention relates to a human fungal disease treating composition being characterized by comprising haterumalide A, B, E and/or X or a derivative thereof in admixture with a pharmaceutically acceptable carrier or diluent. The composition may be-formulated into different administration forms, as is well known in the art.

A fourth aspect of the invention relates to a method for the preparation of haterumalide A, B, E and/or X being characterized by cultivating, under haterumalide producing conditions, the bacterial strain Serratia plymuthica A 153, and optionally isolating and purifying haterumalide A, B, E and/or X from the culture broth.

According to a preferred embodiment of the method for preparing the haterumalides, a cell-free supernatant of the culture broth of strain A 153 is extracted using ethyl acetate, the ethyl acetate extract and the aqueous phase extract are collected and the resulting material is evaporated to dryness. A solution of the ethyl acetate and aqueous phase extracts are then subjected to a separation procedure by HPLC.

A fifth aspect of the invention relates to a method of controlling plant losses caused by pathogenic fungi and/or of controlling weeds, and comprising the introduction of an antipathogenically or weed suppressing effective dose of the bacterial strain A153 or of haterumalide A, B, E and/or X and/or a derivative thereof or a plant disease or weeds controlling composition containing said bacterial strain or compound(s) into the environment where the disease or weed is to be suppressed. Said bacterial strain, compounds and compositions can be applied to seeds, seedlings, plant vegetative propagation units, crops, plants or plant parts or soil, using application methods and apparatus known in the art.

Quantities of the haterumalide-producing strain Serratia plymuthica A 153 are best obtained by a fermentation process that comprises inoculating a sample of a pure culture of the strain into a liquid shake culture or in a fermentor containing a suitable fermentation medium. The strain may also be grown on a sterile surface, e.g. an agar surface, and when grown out, the cells may be suspended in liquid media known in the art. Growing media may in principle be any bacterial growth medium known in the art. The fermentation is carried out until a sufficient concentration of cells, e.g. about 10⁹-10¹¹ cfu (colony forming units)/ml for liquid cultures, is obtained. The so obtained fermentation broth or bacterial suspension is then centrifugated and the supernatant collected. Optionally, the supernatant is chromatographed on a reverse phase column to isolate and purify the haterumalides of the invention.

In one type of treatment the bacterial cells in the fermentation broth may be centrifuged down and the resulting broth or supernatant, containing the active metabolites, may be used for weed and/or fungal growth control purposes, with or without prior concentration. Bacterial suspension and fermentation broth may also be homogeneously mixed with one or more compounds or groups of compounds known in the art, provided such compounds are compatible with the bacterial strain. Suitable compounds may be ionic and non-ionic surfactants as well as other adjuvants, known in the art, e. g. wetting and spreading agents, stickers, antievaporants, humectants, activators and penetrators. More specifically such compounds may be carbon source nutrients or compound bacterial nutrients, metal salts, salts from fatty acids, fatty acid esters, or other compounds acting as synergists including herbicides, fungicides, insecticides, bactericides, other biocontrol agents as well as their fermentation broth and the like.

Bacterial suspensions and fermentation broth may also be freeze dried prior to or after being mixed with suitable compounds and the resulting product used for weed and/or fungal growth control.

Likewise, the supernatant or the haterumalide A, B, E and/or X as such may be formulated into plant disease and/or weed controlling compositions. Thus, the supernatant or haterumalide may be homogeneously mixed with one or more compounds or groups of compounds known in the art, provided such compounds are compatible with the haterumalide(s) in question. Suitable compounds may be powdery additives or solid carriers, such as talcum, kaolin, bentonite or montmorillonite, wettable powders known in the art, metal salts, salts from fatty acids, fatty acid esters, ionic or non-ionic surfactants, plant nutrients, plant growth regulators, other fungicides, insecticides, bactericides, other herbicides and the like. The supernatant containing the haterumalides may also be dried or freeze-dried prior to or after being mixed with suitable compounds and the resulting product used for plant protection.

Bacterial preparations may be applied in any manner known for controlling weeds and/or fungal growth. Atomizing, dusting, scattering of granules or drenching may be chosen in accordance with the intended object and the prevailing circumstances. Advantageous rates of application when used in plant production are normally from 10¹³ to 10¹⁵ cfu/ha or a corresponding amount of bacterial metabolites.

Haterumalide A, B, E and X and compositions containing them may be applied in any manner known for treating seeds, vegetative propagation units, plants, crops and soil with biocontrol active substances. Spraying, atomizing, dusting, scattering, pelleting, dipping or pouring may be chosen in accordance with the intended objective and the prevailing circumstances.

For further information of suitable choice of formulation and application methods, reference is made to Rhodes (1993).

It is preferred to apply haterumalide A, B, E and X or a derivative thereof or a composition containing them to seeds or soil when the purpose is to protect plants or crops from fungal diseases. For controlling weeds in crops, it is preferred to apply the haterumalides or a haterumalide-containing composition by spraying on the crops or on the soil.

In the experimental section to follow, we report on the isolation, preservation and biological activity of the novel bacterial strain A153 as well as on the isolation, structure, characterization and biological activity of the fungal disease and weed suppressing haterumalides of the invention. Said section is offered to further illustrate the invention but not to limit its scope.

### EXPERIMENTAL SECTION

A. Materials and Methods

### EXAMPLE 1

### Isolation of the Seriatia plymuthica Strain A153

The dug up roots of wheat plants (Triticum aestivum) were washed in sterile tap water to remove adhering soil. From a young root a piece, 2-3 cm long, was cut out and handled under sterile. conditions. The piece was taken from the region close to the root tip Small cuts were made in the root piece with a flamed scalpel. The root piece was then rubbed against the surface of TSA 10 agar (Tryptic Soy Agar, Oxoid Ltd., 10 g/liter). After bacteria had grown out on an agar plate incubated at 15° C. for nine days, an A153 colony was picked and the bacterium was pure cultured onto TSA 10 at 5° C. for five days. C. f. Åström and Gerhardson, 1988.

The strain was tentatively identified using the API NE 20 test (API System, Ltd., France) and further identification was confirmed by DSMZ (Deutsche Sammlung von Mikroorganismen und Zellkulturen GmbII, Germany).

### EXAMPLE 2

### Preservation of Strain A153

The pure culture was deep freezed in small ampoules at −70° C. As freeze supports were used 10% glycerol in tap water, pH adjusted to 7.15 after autoclaving. After freezing at −70° C., the ampoules were moved to −20° C.

For long term preservation the isolate was freeze-dried. After growing for 48 hour on TSA 10 agar (Tryptic Soy Agar, Oxoid Ltd., 10 g/liter), the bacterial lawn was scraped off the agar surface, mixed with a freeze drying support (Dextran T 70, Pharmacia Fine Chemicals Ltd., 50 g; Na-L-glutamate, Kebo AB, 50 g; and 1000 ml of distilled water), poured into small ampoules (20 ml) and put in a Hetosicc freeze drier (Heto Ltd., Denmark) for 24 hours. After freeze drying the ampoules were gas tightly sealed with rubber stoppers and stored at 4° C.

### EXAMPLE 3

### Proliferation of Strain A153

Fifty per cent strength TSB medium (TSB, 15 g Tryptic Soy Broth (Difco Ltd) and 1000 ml deionized H₂O) were used to culture the isolate A153. The cultures, 50 ml, 250 ml and 1000 ml, respectively, were grown in 100 ml, 500 ml and 2000 ml Erlenmeyer flasks and were initiated either by transferring a single colony from the TSA plate or by transferring 0.1 ml of a 24-hours old starter inoculum per 5 ml of fresh medium. The cultures were incubated on a rotary shaker (120 rev min⁻¹) for 48 hours at 20° C. in darkness and the cell densities obtained were then approximately from 1 to 1.5×10⁹ cfu ml⁻¹. Only 48-hours old cultures were used during purification procedures.

### EXAMPLE 4

### Fungal Isolates

The fungal isolates of Aspergillus fumigatus Fres. (A. fumigatus) and Microsporum canis var. canis (M. canis) were kindly provided by Department of Microbiology, the isolate of Heterobasidion annosum by Department of Forest Mycology and Pathology, the isolates of Fusarium culmorum (W. G. Smith) Sacc. (F. culmorum), Pythium spp. ( ) and Sclerotinia sclerotiorum (Lib.) de Bary (S. sclerotiorum) by Plant Pathology and Biocontrol Unit, all at SLU, Uppsala, Sweden. The isolate of Fusarium oxysporum Schlecht.: Fr. (F. oxysporum) was kindly provided by Findus R & D Centre Bjuv AB, Sweden and the isolate of Candida albicans (C. albicans) by the Laboratory of Clinical Microbiology, Centre of Laboratory Medicine, University Hospital, Uppsala, Sweden. All fungal isolates, except for the isolate of C. albicans, were maintained on Malt Extract Slants (ME, 50 g Malt Extract, 16 g Agar in 1000 ml distilled H₂O, Difco Ltd.) at 4° C. The isolate of C. albicans was maintained as a deep frozen (−70° C.) stock culture.

### EXAMPLE 5

### Fungal Spore Suspensions

The spore suspensions of test fungi and the stock culture of C. albicans were used to detect antifungal metabolites produced by the S. plymuthica A 153 during purification procedures and to test the minimal inhibitory concentrations needed by purified metabolites for a total inhibition of spore germination. To obtain the A. fumigatus conidiospores, the fungus was grown on ME slants for about 2 weeks at 28° C. The F. culmorum and F. oxysporum conidia, the H. annosum basidiospores, the M. canis macroconidia were produced by inoculating fungal plugs on Corn Meal Agar plates (CMA, Difco Ltd.), Hagem Agar plates (5 g glucose, 5 g ME, 0.5 g NH₄NO₃, 0.5 g KH₂PO₄, 0.5 g MgSO₄×7H₂O, 20 g Agar in 1000 ml deionized H₂O) and Oat Meal Agar plates (60 g oat meal, 12.5 g Agar in 1000 ml deionized H₂O), respectively, for a period of 5 to 10 days at 25° C. Ascospores of S. sclerotiorum were collected during their dispersal from apothecia by entrapping them on the surface of a water agar (12 g Bacto Agar (Difco Ltd)), 1000 ml H₂O). Mature spores were then scraped off or washed out from the surface of the agar plates, with 0,5% Bacto Peptone (Difco Ltd) supplemented with 0.01% Tween 80. The P. ultimum sporangia were obtained by. growing the fungus on grass blades (Martin, 1992). The C. albicans stock culture of 1.0×10⁶ cfu ml⁻¹ was prepared from a 24-hours old culture grown on Yeast Malt Agar plates (YM Agar, Difco Ltd) at 37° C. by suspending it in 0.5% Bacto Peptone and adjusting the optical density of this culture to an absorbance of 0.5 at 600 nm. The spore concentration was counted by using a Bürker chamber, and stock spore suspensions were stored at −70 ° C. for further use. The spore concentrations in respective stock suspensions, as well as the spore concentrations and media to culture fungi in a microtiter plate assay, are listed in Table 2 below.

TABLE 2
The spore concentration of stock and test fungal
suspensions and the culturing media used.

| | Spore concentration (ml⁻¹) | | |
|---|---|---|---|
| Fungus | Stock suspension | Test suspension | Culturing media |
| A. fumigatus | 4.8 × 10⁸ | 4.8 × 10⁴ | MRSB^{x} |
| C. albicans | 1.0 × 10⁶ | 1.0 × 10⁴ | YMB^{y} |
| F. culmorum | 0.9 × 10⁷ | 0.9 × 10⁴ | MEB^{z} |
| F. oxysporum | 1.8 × 10⁶ | 1.8 × 10⁴ | MEB |
| H. annosum | 1.7 × 10⁶ | 3.4 × 10⁴ | MEB |
| M. canis | 2.5 × 10⁵ | 2.5 × 10⁴ | MEB |
| P. ultimum | 1.6 × 10⁵ | 1.6 × 10⁴ | MEB |
| S. sclerotiorum | 2.8 × 10⁵ | 2.0 × 10⁴ | MEB |

^{x}MRSB (62 g de Man Rogosa Sharpe Broth (Oxoid) in in 1000 ml H₂O)
^{y}YMB (3 g Bacto Yeast Extract, 3 g Bacto Malt Extract, 5 g Bacto Peptone, 10 g Bacto Dextrose (all Difco Ltd.) in) in 1000 ml H₂O)
^{z}MEB (30 g Malt Extract (Difco Ltd) in 1000 ml H₂O)

### EXAMPLE 6

### Sclerotinia sclerotiorum Isolates and Production of Sclerotia

One S. sclerotiorum isolate was derived from a sclerotium collected from an oil seed rape plant (Sweden). One isolate was derived from a sclerotium collected from a carrot (Sweden) and two isolates were derived from sunflowers (ND, USA). The sclerotia were surface sterilized in 70% EtOH for 2 minutes, rinsed in sterile distilled water, bisected and one of the two sclerotial halves were placed on a Potato Dextrose Agar (PDA, Oxoid Ltd.) plate with the freshly cut surface towards the agar. The inoculated plates were incubated for 3 to 5 days at 20° C. in darkness. Larger amounts of sclerotia were produced on autoclaved wheat, and were preconditioned for apothecial development before being used in tests (Thaning and Nilsson, 2000). For long term storing, the sclerotia were surface sterilized in 70% EtOH, rinsed in sterile de-ionized water and placed in open plastic bags at 1.5° C.

### EXAMPLE 7

### Bioassay Procedures

Suppression of Spore Germination—Microtiter Plate Assay 1

To follow the activity during purification, HPLC-fractions to be tested were directly collected to 96-well microtiter plates by using a fraction collector (FC 204, Gilson), and mainly the activity to inhibit the germination of F. culmorum conidia and S. sclerotiorum ascospores was tested. In order to test the minimal inhibitory concentrations of purified metabolites needed for a total inhibition of spore germination of all test fungi, the appropriate sample volume out of the haterumalide stock solutions of 100 μg ml⁻¹, 10 μg ml⁻¹ and 1 μg ml⁻¹ in acetonitrile was pipetted into the wells. The solvents were always evaporated, and one hundred μl of spore suspension, suspended in the appropriate media as listed in Table 2, were then distributed into each well. Depending on the fungus tested, the spore germination was monitored after 24 to 96 hours of incubation at 25° C. in darkness except for A. fumigatus and C. albicans which were incubated at 28 ° C. and 37 ° C. Controls were spores suspended in respective culturing media and culturing media only. All tests were done in triplicate and repeated at least twice.

Suppression of Apothecial Formation—Microtiter Plate Assay 2

Twenty four-well microtiter plates were used to test the effect of the purified metabolites on apothecial formation in a concentration range between 0.01 μg ml⁻¹ and 100 μg ml⁻¹. To obtain respective concentrations of tested metabolites, the appropriate volume out of the respective metabolite stock solutions of 100 μg ml⁻¹, 10 μg ml⁻¹ and 1 μg ml⁻¹, dissolved in acetonitrile, was distributed into the wells and the solvent was then evaporated. Thereupon, the metabolites were dissolved in sterile H₂O and one, or in some tests two, preconditioned sclerotia were placed into each well. Tests were done in triplicate and controls were sclerotia placed in sterile water only.

Suppression of Apothecial Formation—Petri Plate Bioassay 1

This bioassay was used to screen bacterial isolates for their effects on apothecial formation and to assay the bioactivity during the first steps of purification procedure of an active metabolite. In this bioassay, ten evenly sized (5 to 8 mm) and preconditioned S. sclerotiorum sclerotia were placed on the top of one cm layer of a non-sterile commercial peat soil mixture (Hasselfors AB, Sweden; 80% peat and 20% sand) in a Petri plate (9 cm in diameter). They were then covered with an additional centimeter of soil. Five ml of tested bacterial cultures or other respective treatments were evenly applied over the soil surface of each Petri dish. The water potential of the peat soil after bacterial application was adjusted to about −0.1 bars. As a complementary screening, sclerotia were placed on moist filter paper and treatments were added directly on sclerotia. After the application of the tested material, the Petri plates were sealed with parafilm, placed at a distance of 40 cm under fluorescent tubes (True-Lite 58 W, Rätt Ljus AB, Bromma, Sweden and Duro-Test International Inc., Fairfield, N.J., USA) at a temperature of 18 ° C. and a photoperiod of 16 hours with 50 μE of light m⁻²s⁻¹. The suppressive effect on carpogenic germination and apothecial formation of the treated sclerotia was estimated by counting the number of apothecia after 10 and 17 days, respectively.

Suppression of Apothecial Formation—Petri Plate Bioassay 2

This bioassay was used during HPLC-guided purification procedure for active metabolites. Ten preconditioned sclerotia were placed in a 5 ml layer of a steam-sterilized natural sandy soil in a Petri plate (4 cm in diameter), and covered with additional 2 ml of the same soil. The steam-sterilized natural sandy soil was used in this assay for two reasons: i) to reduce possible effects of interfering microorganisms and, ii) sandy soil may contain lower concentrations of toxic substances than peat soil after steam sterilization. Smaller sized Petri plates were used to reduce the amount of product needed for the experiments. For testing the suppression of apothecial formation, the HPLC fractions were always diluted to a concentration corresponding to the bacterial culture with water supplemented with, at most, 1% MeOH. The test was always repeated with fractions 4 and 10 times more concentrated than the bacterial solutions to counter the possible dilution caused by the cutting of fractions. One ml of tested sample was then applied over the soil surface. The Petri plates were then treated and the results evaluated as described in bioassay 1. Appropriate controls were always included.

### EXAMPLE 8

### Viability of Recovered Scierotia

In order to test viability of sclerotia treated with the purified metabolites, sclerotia, which did not form apothecia, were retrieved, four weeks after a test start, from the test Petri plates or from microtiter plates. The sclerotia were placed in 100 ml of deionized water for 10 minutes and subsequently. washed with running deionized water for 10 minutes. They were then transferred onto moistened filter paper in sealed Petri dishes and placed under light conditions suitable for induction of apothecial formation. After one and two weeks, sclerotia were observed for apothecial formation. The sclerotia, which did not produce apothecia, were subsequently tested for myceliogenic germination. These sclerotia were surface disinfected in 70% ethanol for 2 minutes, rinsed in sterile deionized water, bisected, transferred to PDA in Petri dishes, and observed for mycelial growth after 5 days.

### EXAMPLE 9A

### Purification, HPLC Separation and Structure Elucidation of the Active Metabolites

The cell free supernatant of the culture broth of the strain A 153, obtained after centrifugation (Sorvall RC 5B centrifuge, 20,000×g, 20 min, 4° C. or 15,000×g 30 min, 4° C.), was used to purify the active metabolites. The 1.8 l of supernatant were first continuously extracted using 0.5 l of recirculating ethyl acetate (EtOAc), for 18 hours at 105° C. The ethyl acetate extract (EtOAc extract) and the aqueous phase extract (AqPh. extract) were collected and the resulting material was then evaporated to dryness. The EtOAc and AqPh extracts from two extractions (3.6 l) were finally dissolved in 2 ml MeOH/H₂O (40/60, v/v) and 13 ml MeOH/H₂O (50/50, v/v), respectively, and stored at −20° C. for the following analysis.

The EtOAc extract was then separated by HPLC by four injections on a Prep Nova-Pak C18-column, 25×100 mm (Waters). Five-milliliter fractions were collected and after monitoring antifungal activity, the active fractions were pooled together, evaporated and dissolved in acetonitrile. The active compounds were finally separated after additional fractionating on a Prep Nova-Pak C18-column, 25×100 mm (Waters). An analytical Nova-Pack C18-column, 8×100 mm (Waters) and a HyPurity Advance C8 column, 4.6×100 mm, (Hypersil Division, ThermoQuest, Needham, Mass.) were used to monitor the retention time of the active substances and to separate fractions into microtiter plates. All systems were operated isocratically with an eluent consisting of a mixture of ACN/H2O (40:60) at a flow rate of 5.0 ml min⁻¹ (NovaPack preparative column), 1.0 ml min⁻¹ (NovaPack analytical column) and 0.8 ml min⁻¹ (Hypurity Advance column), respectively. The components were monitored with a diode array detector at the UV absorbance range between 190 and 400 nm. The absorbance data at 200 nm were used for all calculations. During the purification procedures, the antifungal activity of HPLC-separated fractions was monitored, by testing the inhibition of F. culmorum and S. sclerotiorum spore germination with the microtiter plate assay 1 described in Example 7.

NMR data were collected using a Bruker DRX-600 NMR spectrometer equipped with a 2.5 mm probe. Spectra were recorded with an irradiation of 600,13 MHz for proton and 150.90 MHz for carbon spectra. Standard pulse sequences were used with mixing times of 40 and 100 ms for HMBC, 1.72 ms for HSQC-DEPT and 80 ms for NOESY spectra. Standard samples were made by dissolving 1-3 mg of the compounds analysed in 120 l deuterated solvent. Chemical shift values were determined relative to standard values for solvent peaks.

After the EtOAc extract fractionation on the analytical Nova-Pack C18-column, the inhibition of spore germination of F. culmorum and S. sclerotiorum was detected within four well-separated regions. Six substances showing antifungal activity were finally obtained in a two-step HPLC purification procedure using the Prep Nova-Pak C18-column. Four of the active substances showed a maximum UV absorption of about 193 nm and two other compounds had a maximum UV absorption of about 193, 209 and 249 nm, compound five, and 215 and 303, compound 6. On the basis of mass- and NMR-spectroscopic data, three of the first metabolites were determined to be identical to a compounds isolated separately by researchers in Japan and the United States. The first of three similar compounds has been given the names haterumalide A and oocydin A by the two groups, respectively. For the purposes of this invention we have chosen to use the name haterumalide A. The two other were haterumalide B and E described by researchers in Japan. The fourth compound was found to be similar to haterumalides A, B and E and has been named haterumalide X. Compound five was identified to be pyrrolnitrin and compound six to be 1-acetyl-7-chloro-1-H-indole. The retention time of these six pure substances, eluted with a mixture of ACN/H2O (40/60 v/v) supplemented with 0.1% acetic acid on a HyPurity Advance Column was detected to be 7.0 min (haterumalide A), 8.5 min (haterumalide B), 8.7 min (haterumalide X), 8.8 min (haterumalide E), 12.5 min (pyrrolnitrin) and 6.9 min (1-acetyl-7-chloro-1-H-indole), respectively.

The NMR data for haterumalide A, B, E and X are listed in Table 1 above.

### EXAMPLE 9B

### HPLC Separation and Structure Elucidation of Haterumalide A

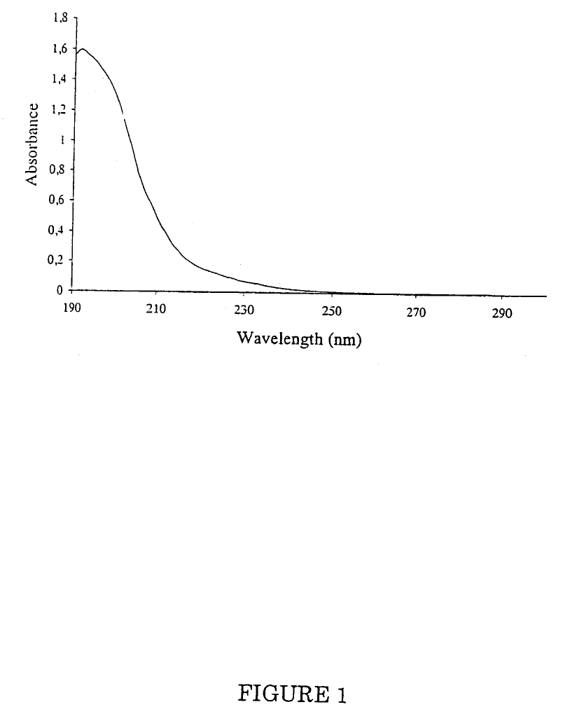

An active metabolite, which suppressed both apothecial formation and ascospore germination, was purified from an EtOAc extract of the cell free supernatant of the culture of S. plymuthica strain A 153 in a two step HPLC procedure by eluting with a mixture of ACN/H2O (40:60). In a first step of HPLC separation, the activity was detected after testing the fractions, which were collected between 6.5 and 8.5 min, in a region where several other metabolites were also eluted. The active metabolite was finally eluted as a single peak with a maximum UV absorption at 193 nm after additional HPLC separation of pooled active material. The retention time for this compound, when eluted on a HiPurity Advance column with a mixture of ACN/H2O (40:60) supplemented with 0.1% acetic acid, was at 5.3 min. On the basis of mass- and NMR-spectroscopic data (Table 3 below), the isolated metabolite was determined to be identical to a compound isolated separately by researchers in Japan (Takeda et al., 1999) and the United States (Strobel et al., 1999). This compound has been given the names haterumalide A and oocydin A by the two groups respectively. For the purposes of this article we have chosen to use the name hateramalide A. The molecular weight of haterumalide A is 470.17. Its UV spectrum with λ max at 191.5 nm is shown in FIG. 1. The extinction coefficient (ε) was 75000. By applying the above described purification procedure, a total of approximately 1.45 mg of haterumalide A was recovered from 1 liter of culture supernatant.

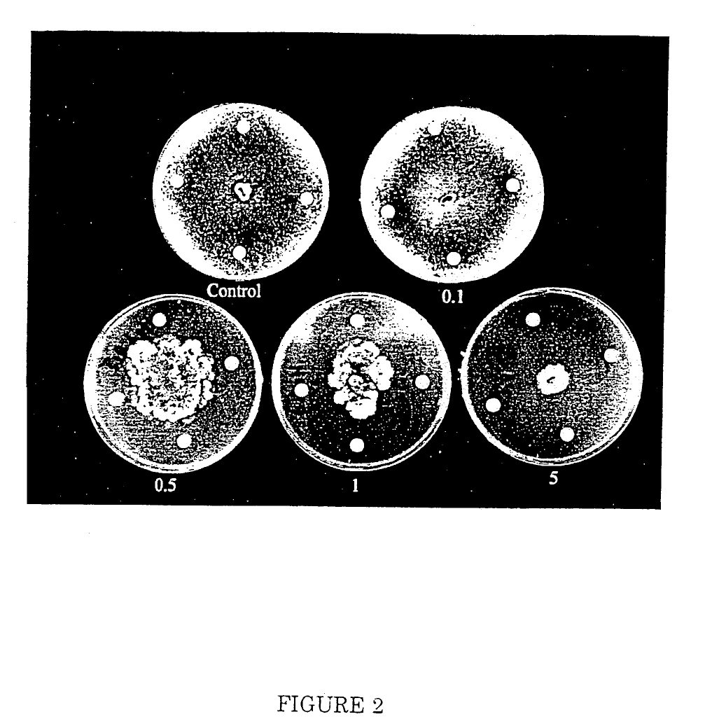

The MIC of haterumalide A needed to totally inhibit the apothecial formation on sclerotia was estimated between 0.2 and 0.5 μg ml⁻¹ haterumalide per one sclerotium (5-8 mm), and a partial inhibition of apothecial formation was observed at the range of 0.05 to 0.1 μg ml⁻¹ (FIG. 2). Retrieved sclerotia, that had been treated with 0.5 and 1 μg ml⁻¹ of haterumalide A, produced apothecia when replaced on a moistened filter paper, whereas treatment with higher concentrations of haterumalide A (3 to 10 μg ml⁻¹) resulted in total inhibition of carpogenic germination. Sclerotia treated with 3 and 5 μg ml⁻¹ produced mycelium when additionally replaced on PDA plates, whereas no mycelial growth was observed after applying 10 μg ml⁻¹ of this metabolite. The haterumalide A MIC totally inhibiting ascospore germination (1.96×104 ml⁻¹) was 0.8 μg ml⁻¹, and a partial inhibition was seen also in the concentration range of 0.1 to 0.7 μg ml⁻¹.

TABLE 3
Comparison of NMR data obtained for haterumalide A.

| | Data from Takada et al., 1999 | | Data from Strobel et al., 1999 | | Data acc. to the invention | |
|---|---|---|---|---|---|---|
| Position^{a} | δC | δH | δC | δH | δC | δH |
| 1 | 169.5 | | 169 | | 169.0 | |
| 2 | 38.9 | 2.77 | 38 | 2.78 | 38.1 | 2.82 |
| 2 | | 2.82 | | 2.82 | | 2.85 |
| 3 | 68.7 | 5.79 | 68 | 5.79 | 67.9 | 5.81 |
| 4 | 134.7 | | 134 | | 134.4 | |
| 5 | 130.9 | 5.70 | 130 | 5.70 | 130.3 | 5.70 |
| 6 | 35.8 | 2.45 | 35 | 2.45 | 36.9 | 2.50 |
| 6 | | 3.50 | | 3.48 | | 3.52 |
| 7 | 126.9 | 5.30 | 127 | 5.31 | 126.1 | 5.28 |
| 8 | 133.2 | | 132 | | 133.5 | |
| 9 | 35.5 | 2.28 | 29 | 2.28 | 34.7 | 2.27 |
| 9 | | 2.45 | | 2.47 | | 2.48 |
| 10 | 29.0 | 1.38 | 28 | 1.39 | 28.1 | 1.41 |
| 10 | | 2.28 | | 2.30 | | 2.32 |
| 11 | 78.1 | 3.93 | 79 | 3.94 | 77.3 | 3.97 |
| 12 | 38.7 | 1.52 | 38 | 1.52 | 38.1 | 1.52 |
| 12 | | 2.09 | | 2.09 | | 2.11 |
| 13 | 76.7 | 5.29 | 77 | 5.30 | 75.9 | 5.32 |
| 14 | 84.6 | 3.89 | 85 | 3.90 | 83.6 | 3.95 |
| 15 | 66.5 | 4.52 | 66 | 4.53 | 66.5 | 4.58 |
| 16 | 129.8 | 5.34 | 130 | 5.37 | 130.0 | 5.41 |
| 17 | 136.8 | | 135 | | 133.2 | |
| 18 | 47.8 | 2.97 | 47 | 3.01 | 45.2 | 3.03 |
| 18 | | 3.01 | | 3.04 | | 3.06 |
| 19 | 179.2 | | 175 | | 174.8 | |
| 20 | 18.5 | 1.88 | 19 | 1.88 | 18.1 | 1.90 |
| 21 | 17.4 | 1.82 | 18 | 1.82 | 16.9 | 1.85 |
| 22 | 171.3 | | 171 | | 170.6 | |
| 23 | 20.9 | 2.02 | 21 | 2.02 | 20.9 | 2.05 |

^{a}Assignments based on COSY, HSQC-DEPT and HMBC spectra.

B. Fungal Growth Control

### EXAMPLE 9

### Effects of A153 on Spore Germination of a Number of Plant Pathogenic Fungi

Two hundred milliliters of TSB-supernatant of the isolate A153 and 150 milliliters of ethyl acetate were used to extract and purify the supernatant by using continuous extraction for 16 hours. Two fractions, ethyl acetate extract (EtOAc extract) and water phase extract (AqPh extract) were separated during extraction. The EtOAc extract was then evaporated to dryness and dissolved in 2 ml of 99% methanol and the AqPh extract was evaporated and concentrated to the volume of 20 ml (10 times).

The effect of both extracts on germination of fungal spores of Aspergillus fumigatus, Fusarium culmorum, Fusarium oxysporum, Heterobasidion annosum, Microsporum cannis, Pythium ultimum and Sclerotinia sclerotiorum was tested on microtiter plates. The volumes from 50 μl, to 1 μl of EtOAc extract and from 100 μl to 5 μl of AqPh extract were respectively dispensed to wells on microtiter plates and then evaporated to dryness One hundred microliters of fungal spores (10⁴-10⁵ spores/ml) in Malt extract medium (Difco) and/or in MRSB medium (Oxoid) were distributed to wells with dried tested extracts. Plates were then incubated for a period of 48 to 96 hours at 25 C. Results of inhibition of spore germination were evaluated in comparison to negative control (only medium) and to positive control (medium with fungal spores).

TABLE 4
The inhibition of Aspergillus fumigatus, Fusarium culmorum, Fusarium
oxysporum, Heterobasidion annosum, Microsporum
cannis, Pythium ultimum and Sclerotinia
sclerotiorum spore germination by EtOAc extract
and AqPh extract of the isolate A 153. Results are shown as
lowest concentrations of extracts needed for total inhibition
of spore germination.

Concentrations of extracts

| Tested fungus | EtOAc extract (C) | AqPh extract (C) |
|---|---|---|
| Aspergillus fumigatus | 25 | >10 |
| Fusarium culmorum | 1 | 2.5 |
| Fusarium oxysporum | 2.5 | 10 |
| Heterobasidion annosum | 1 | 2.5 |
| Microsporum cannis | 1 | >10 |
| Pythium ultimum | 0.5 | 0.5 |
| Sclerotinia sclerotiorum | 35 | >10 |

### EXAMPLE 10

### Antifungal Spectra of Haterumalide A, B and E

The activity of haterumalide A, B and E, and of pyrrolnitrin to inhibit the spore germination of A. fumigatus, F. culmorum, F. oxysporum, H. annosum, M. canis, P. ultimum and S. sclerotiorum as well as the inhibition of the C. albicans growth was tested by using the microtiter plate assay 1 described in Example 7. The effect on apothecial formation of S. sclerotiorum sclerotia was assayed by using microtiter plate assay 2 described in Example 7.

The MIC of haterumalides A, B and E and the MIC of pyrrolnitrin needed for total inhibition of spore germination of various fungi and apothecial formation of sclerotia in our microtiter plate systems are shown in Table 5 below. The spore germination of all tested fungi was detected in the presence of haterumalides A, B and E and in the presence of pyrrolnitrin, whereas in a bioassay to inhibit apothecial formation of sclerotia only haterumalides A, B and E were tested. The three haterumalides, at low concentrations, effectively inhibited the spore germination of F. culmorum, Pythium spp. and S. sclerotiorum and, at slightly higher concentrations, were also effective against A. fumigatus and F oxysporum. Interestingly, their effectiveness to inhibit apothecial formation of sclerotia was independent on the form tested, whereas the inhibition of spore germination was variable and depended on the form of haterumalide tested. In general, haterumalides A and B were more effective than haterumalide E. When comparing the effectiveness of haterumalides A and B with that of pyrrolnitrin, lower concentrations of haterumalides A and B were needed to totally inhibit germination of F. oxysporum, H. annosum and S. sclerotiorum spores and higher to inhibit A. fumigatus, F. culmorum spore germination. The growth of C. albicans was not totally inhibited by any of tested substances at concentrations up to 100 μg ml ⁻¹.

TABLE 5
The MIC of haterunalides A, B and E and the MIC of pyrrolnitrin
needed to totally inhibit spore germination of
A. fumigatus, F. culmorum, F. oxysporum, H. annosum, M. canis,
P. ultimum and S. sclerotiorum and to totally inhibit apothecial
formation of S. sclerotiorum sclerotia tested in microtiter plate systems.

| | Minimal Inhibitory Concentration (MIC, μg ml⁻¹) | | | |
|---|---|---|---|---|
| Fungus | Haterumalide A | Haterumalide B | Haterumalide E | Pyrrolnitrin |
| Inhibition of spore | | | | |
| germination | | | | |
| A. fumigatus | 5 | 13 | 15 | 3 |
| F. culmorum | 2 | 1 | 5 | 0.06 |
| F. oxysporum | 0.8 | 0.8 | 25 | 20 |
| H. annosum | 35 | 20 | <50 | 50 |
| M. canis | 15 | 40 | <75 | 1 |
| P. ultimum | 0.4 | 1 | 8 | <50 |
| S. sclerotiorum | 0.8 | 3 | 3 | 10 |
| Inhibition of apothecial | | | | |
| formation of sclerotia | | | | |
| S. sclerotiorum | 0.5 | 0.5 | 0.5 | <100 |

### EXAMPLE 11

### Suppression of S. sclerotiorum Mycelial Growth

Dual cultures of the isolate A 153 and S. sclerotiorum were inoculated on PDA plates (9 cm in diameter). An agar plug with actively growing S. sclerotiorun mycelium was placed in the center of plate and the bacterial isolate was inoculated at four equidistant places, each 1 cm from the edge of the plate. Inhibition zones were measured after 4 days incubation at 18° C. in darkness and the effect was compared to controls with only S sclerotiorum. In addition, the possible effects of bacterial treatment on mycelial development were observed under a microscope at 200-X magnification.

For testing effects of haterumalide A, halves of surface sterilized and bisected sclerotia were placed in the center PDA plates (9 cm in diameter) to initiate mycelial growth of S. sclerotiorum. The metabolite, at a concentration from 0.05 to 5 μg ml⁻¹ diluted in MeOH/H₂O (1:1), was then spotted directly onto sterile antimicrobial susceptibility test discs (Oxoid Ltd) located at four equidistant places, giving a total of 0.2 to 20 μg ml⁻¹ compound per one Petri plate. The treatments were performed in triplicate. Zones of inhibition of mycelial growth were measured after 4 days and reduction of mycelial growth was calculated as a percentage of MeOH/H₂O controls.

Strong inhibition of S. scierotiorum hyphal growth by the isolate A 153 was observed in dual cultures where the average radial colony growth of the fungus did not exceed 10 mm in diameter when colonies on control plates had a radius of 85 mm. Under the microscope, hyphal tips were observed to be bent and afterwards fungal cell walls showed disruption, followed by leakage of cytoplasm.

At lower concentrations (0.05 to 0.1 μg ml⁻¹ per disc), haterumalide A did not inhibit the mycelial growth of S. sclerotiorum, (FIG. 2). The mycelial growth of fungus was however increasingly inhibited, from 47 to 84%, in the presence of haterumalide A at concentrations from 0.5 to 5 μg ml⁻¹ per disc. Hyphal tips were bent as described above but in this case we observed no cell disruption or leakage.

### EXAMPLE 12

### Apothecial Suppression by Various Preparations of the S. plymuthica A 153 Broth Culture

Using the suppression of apothecial formation, Petri plate bioassay 1 (Example 7), the following preparations of the culture of the isolate A 153, cell free supernatant, washed cells, cell lysate as well as ethyl acetate and aqueous phase residue extracts of the cell free supernatant, were tested for their effects on apothecial formation. Bacterial cultures were grown as described above. Bacterial cell free supernatants were obtained by centrifugation (Sorvall RC 5B centrifuge, 20 000× g, 20 min, 4° C. or 15 000× g 30 min, 4° C.) and subsequent filter-sterilization (Akafilter, Västra Frölunda, Sweden). Washed bacterial cells were prepared by collecting cell pellets after centrifugation, washing the cells twice with deionized water, centrifuging after washing, and finally suspending the cells to the initial volume with water. To obtain cell lysate, cells were lysed overnight with 4-times acetone volume of the initial culture volume, and acetone was then evaporated to dryness. The remaining material was finally redissolved in 50% MeOH/50% H₂O.

Further preparation was done on the cell free supernatant, which was continuously extracted using 0.2 or 1.8 1 of supernatant, with 0.15 and 0.5 l, respectively, of recirculating ethyl acetate (EtOAc), for 18 hours reflux. The ethyl acetate extract (EtOAc extract) and the aqueous phase extract (AqPh. extract) were collected and the resulting material was then evaporated to dryness. Dried EtOAc and AqPh. extracts were finally dissolved, using minimal volumes of methanol (MeOH), and 1:1 MeOH/H₂O, respectively, and stored at −20° C. for subsequent analysis.

All the preparations were prepared in such a way that their relative concentrations corresponded to those of the bacterial culture, i.e. 1.5×10⁹ cfu ml⁻¹ (non-diluted), 0.75×10⁹ cfu ml⁻¹ (diluted 1:1) and 0.375×10⁹ cfu ml⁻¹ (diluted 1:3), and their apothecial suppressive activity was compared to the effect of such bacterial cultures tested simultaneously. Three Petri plates per treatment were used with sclerotia treated with deionized water, 50, 25 and 12, 5% strength TSB and 1, 0.5 and 0.25% methanol (MeOH) in deionized water, as controls. Inclusion of methanol controls reflected residual concentrations of methanol in the ethyl acetate extratct and aqueous phase. The suppression of apothecial formation after the treatment with various culture preparations was transformed to values of apothecia formed per one sclerotium as a percentage of water control. Sclerotia with suppressed apothecial formation were retrieved and their viability was tested as described above (Example 8).

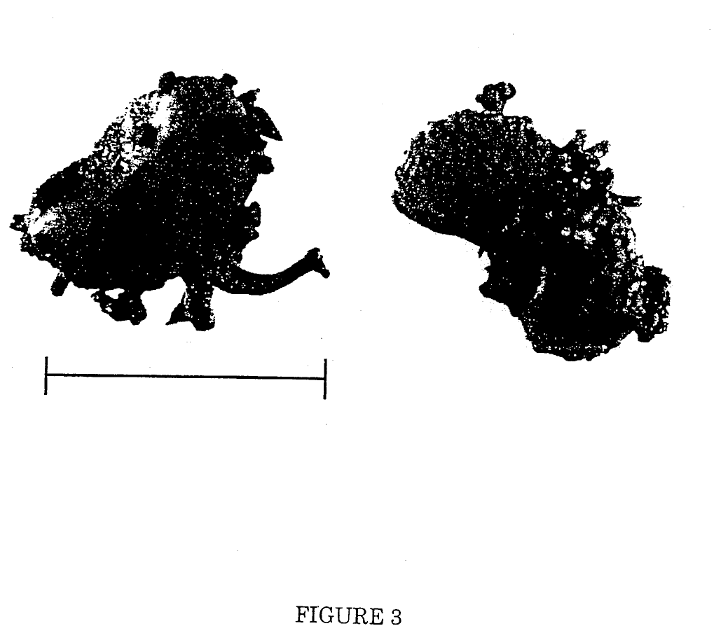

The bacterial culture, its cell free supernatant and the ethyl acetate extract, at relative concentrations corresponding to bacterial culture treatments of 1.5 and 0.75×10⁹ cfu ml⁻¹, significantly reduced the number of apothecia produced by S. sclerotiorum (Table 5 below). Depending on the time when the apothecia were counted, the reductions were from 58 to 96% while a lowering of the relative concentration to 0.375×10⁹ cfu ml⁻¹ resulted in significantly higher numbers of apothecia formed. In cases where stipes did not develop on the treated sclerotia the differentiation of the stipes was highly affected. Their elongation was totally inhibited, the tips became thick and melanized (FIG. 3) and yeliow-brownish droplets were observed on the malformed stipes. The treatment with bacterial culture affected the apothecial formation of sclerotia of all tested S. sclerotiorum isolates. However, on 46% of the retrieved sclerotia that had been treated with bacterial cultures, normal apothecia were formed one week after removing the inhibiting factors by means of washing and surface sterilizing.

Treating the sclerotia with washed bacterial cells, bacterial cell lysate and water phase extract preparations did not significantly affect the formation of apothecia, although the number of apothecia formed was generally somewhat reduced in relation to control treatments. The TSB and MeOH control treatments increased the number of apothecia formed and for the TSB treatment this increase was statistically significant (Table 6).

TABLE 6
The number of apothecia formed per sclerotium in percent of the
water control, after treatment with the broth culture of the isolate
A 153, its preparations: cell free supernatant, washed bacterial cells,
cell lysate, ethyl acetate extract and water phase extract, at three
concentrations tested, and counted after 10 and 17 days. The
concentration of tested preparations was relative this of the
broth culture with respectively 1.5-. 0.75- and 0.375 cfu ml⁻¹.
Number of apothecia formed per one sclerotium treated with only water
was respectively 3.6 after 10 days and 4.9 after 17 days. Means
with the same letters within columns do not differ significantly
(Duncan's multiple range test at P = 0.05, n = 4).

| | Relative concentration of tested preparations | | | | | |
|---|---|---|---|---|---|---|
| | 1.5 × 10⁹ cfu ml⁻¹ | | 0.75 × 10⁹ cfu ml⁻¹ | | 0.375 × 10⁹ cfu ml⁻¹ | |
| Treatment | Day 10 | Day 17 | Day 10 | Day 17 | Day 10 | Day 17 |
| Water (control) | 100 bc | 100 bc | 100 ab | 100 abc | 100 a | 100 ab |
| TSB (control) | 142 a | 141 a | 138 a | 134 a | 104 a | 118 a |
| MetOH control | 126 ab | 105 abc | 133 a | 122 ab | 98 a | 98 ab |
| Bacterial culture | 4 d | 13 d | 18 cd | 42 ed | 19 e | 54 c |
| Cell free supernatant | 6 d | 35 d | 6 d | 24 e | 35 de | 89 abc |
| Washed bacterial cells | 80 c | 88 bc | 57 bc | 65 cd | 55 bcd | 68 bc |
| Cell lysate | 77 c | 79 c | 85 b | 88 bc | 82 ab | 99 ab |
| Ethyl acetate extract | 5 d | 9 d | 17 cd | 38 ed | 45 cde | 70 bc |
| Water phase extract | 75 c | 118 ab | 77 b | 94 abc | 76 abc | 94 ab |

### EXAMPLE 13

### Effects of A153 on Carpogenic Germination of the Fungal Plant Pathogen Sclerotinia sclerotiorum

Laboratory-produced sclerotia of the fungus S. sclerotiorum were placed in a Petri dishes filled with 1 cm peat soil (Hasselfors, commercial mixture with peat (80%)7sand (20%)). After placing ten sclerotia per dish they were covered by another cm. pf peat soil, and were then ready for bacterial treatments. In order to obtain a dose-response curves, five ml of bacterial suspension with increasing concentrations was inoculated per sclerotia-containing Petri dish. In a typical experiment concentrations of 1.6×10⁶, 7.9×10⁶, 1.6×10⁷, 3.1×10⁷ and 6.3×10⁷ bacterial cells/cm were used. Tap water and/or bacterial suspensions not containing A153 were used as control treatments. The soil water potentials in all treatments were between 0 and −0.2 kPa, and after bacterial treatment, the Petri dishes were sealed with paraflim and placed 40 cm under fluorescent light at 18° C., using a photoperiod of 16 hours with 50 μEs m⁻²s⁻¹ of light. The effect of the treatments was read by counting the number of apothecia per Petri dish after 17 days.

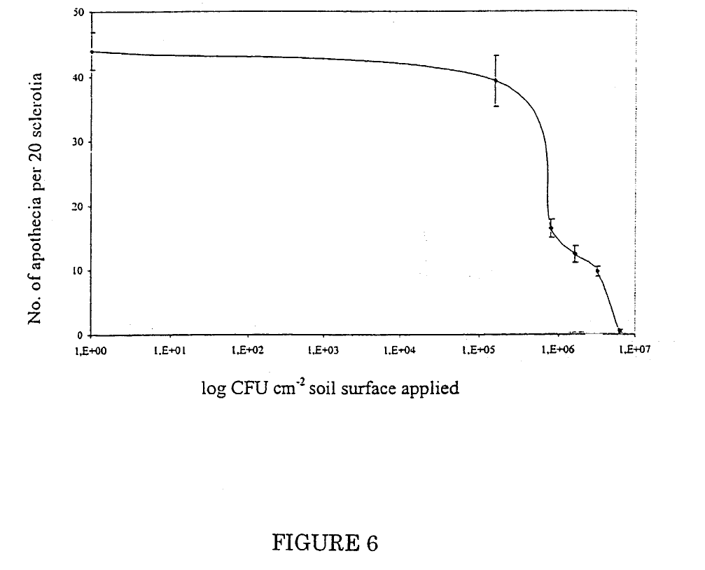

In the dose response test, apothecia developed in the controls starting ten days after incubation. Apothecia were then continuously produced until three weeks after incubation with a maximum number of apothecia observed in the water controls after 17 days. However, an application of bacterial broth culture at a concentration of 6.3×10⁶ CFU cm⁻² totally inhibited apothecial formation, but this strong effect was gradually lost at lower cell concentrations (FIG. 6). The reducing effects on carpogenic germination and apothecial formation were, as also earlier reported (Thaning et al., 2000), found to be dependent on the ontogenetic stage of the sclerotia. Hence, after bacterial application, the stipes developed prior to treatment did not elongate normally, and their tips became thicker and melanized as growth became suppressed. Furthermore, sclerotia without stipes prior to treatment did not germinate carpogenically.

C. Weeds Control

### EXAMPLE 14

### Effects of Strain A153 Against Selected Weeds in Greenhouse Screenings

Bacterial cultures for experimental use were produced by inoculating 24 hours old cultures grown on TSA 10 agar in 15° C., to 50 ml of 50% Tryptic Soy Broth (TSB) contained in conical flasks and then incubating these for 36-48 hours on an orbital shaker at 120-rev min, at 20-22° C., in darkness. For spraying the test plants 5-10 ml of this resulting bacterial suspension was filled in an all glass TLC Reagent Sprayer using compressed air as a propellant source. Bijou bottles were used as reservoir bottles for low spray volumes.

Sowing the weed species Chenopodium album, Stellaria media and Thlaspi arvense in plastic pots with 4 seeds in each pot, produced test seedlings. The pots were 8 cm in diameter and 6 cm high and filled to two thirds with a (unsterilized) commercial peat mixture (Enhetsjord K Normal), mixed with 20% (v/v) sand. When reaching the second-to-fourth leaf stage of growth, or later stages, they were sprayed to runoff, 3-5 ml, depending on plant size and species. Such sensitivity tests were done with at least four replicate pots under three different environmental conditions: greenhouse (10-20, 20-30° C.), in a humid chamber (20-30° C.) at approximately 90% relative humidity and in all cases at a day-length of approximately 12 hours. The experiments were repeated several times on separate dates. After being thus treated the plants were observed for symptoms for up to three weeks beyond the time of showing maximum effect to the agent applied. Optimal bacterial effects were most accurately expressed as percentage mortality and fresh weight reduction. Suboptimal effects, i.e. when test plants were not completely eradicated by bacterial treatments, were also expressed as reductions in shoot length, measured form the soil surface to the shoot meristem. Table 7 below shows the pooled average results obtained in several greenhouse experiments.

TABLE 7
Effect of spraying the bacterial isolate A 153 on leaves
of three weed species in greenhouse experiments.

| | Percentage | Percentage reduction |
|---|---|---|
| Weed species | Mortality | in fresh weight |
| Chenopodium album | 60-80 | 80 |
| Stellaria media | 70-90 | 80-90 |
| Thlaspi arvense | 80-100 | 80-100 |

### EXAMPLE 15

### Greenhouse Experiments Showing A153 Selectivity in Affecting Different Plant Species

The experiments were carried out as described under Example 14 above, but several weed and crop species, as shown in Table 8 below, were treated. Bacterial effects were, furthermore, graded as +++= very strongly susceptible/plant killed; ++ strongly susceptible; += little susceptible (marginal effect); −= totally resistant (no visual effect).

TABLE 8
Selectivity in effects after spraying the bacterial isolate A 153
on shoots of a number of plant species in greenhouse experiments.

| | | |
|---|---|---|
| Plant Family, Genus and Species | | Bacterial |
| treated | Common Name | effects* |

DICOTYLEDONOUS PLANTS

| | | |
|---|---|---|
| Amaranthacea | | |
| Amaranthus retroflexus L. | Redroot Pigweed | ++ |
| Caryophyllaceae | | |
| Stellaria media (L.) Vill | Common Chickweed | +++ |
| Chenopodiaceae | | |
| Chenopodium album | Fat Hen, | +++ |
| | Lambsquarters | ++ |
| Beta vulgaris ssp. vulgaris | Sugarbeet | + |
| Beta vulgaris L. var. cicla | Mangold | ++ |
| Spinacea oleracea L. | Spinach | |
| Compositae | | |
| Matricaria inodora L. | Scentless Chamomile | − |
| Cirsium. arvense (L.) Scop. | Canada Thistle | − |
| Convolvulaceae | | |
| Convolvulus arvensis | Field Bindweed | − |
| Brassicaceae (Cruciferae) | | |
| Brassica napus L. ssp. napus | Oilseed Rape | + |
| Sinapis arvensis L. | Wild Mustard, | + |
| | Charlock | |
| Thlaspi arvense L. | Field pennycress | +++ |
| Fabaceae | | |
| Phaseolus vulgaris | Bean | + |
| Pisum sativum | Pea | + |
| Solanaceae | | |
| Solarnum tuberosum. | Potato | − |
| Violaceae | | |
| Vioala arvensis | Field pansy | + |

MONOCOTYLEDONOUS PLANTS

| | | |
|---|---|---|
| Poaceae (Gramineae) | | |
| Avena fatua | Wild Oats | − |
| Avena sativa | Oats | − |
| Elymus repens | Common couch | − |
| Hordeum vulgare | Barley | − |
| Secale cereale | Rye | − |
| Triticum aestivum | Wheat | − |
| Zea mays | Maize | − |

*+++= very strongly susceptible/plant killed;
++ strongly susceptible;
+= little susceptible (marginal effect);
−= totally resistant (no visual effect)

### EXAMPLE 16

### Effects on a Dicotyledonous Plant of Spraying A 153 in a Barley Crop in Field Experiments

The field experiments, designed as randomized blocks with four repetitions, had plot sizes of 24 m². They were placed in a barley crop undersown with red clover, which in this case functioned as the model, target weed, and were located about 20 km NW of Uppsala on a loamy soils with about 3 per cent humus content. The barley crop was fertilized etc. according to the recommendations in the area, but no weed control was applied.

Bacterial suspensions for spraying was produced as described under Example 14 above, except that instead of using 50 ml of 50% Tryptic Soy Broth (TSB) in each conical flasks, bigger three liter flasks with 1.5 liter TSB broth in each were used. The treated plots were sprayed twice at the barley three-leaf stage and at the barley late four-leaf stage using a conventional hand-operated backpack spayer equipped with a four nozzle-boom at an application volume of 10001/ha and a dose of 10⁹ cfu/ml. Control plots were sprayed with tap water. Care was taken not to spray in strong sunlight and not directly in connection to rain, e. g preferably late in the afternoon on cloudy weather with no wind.

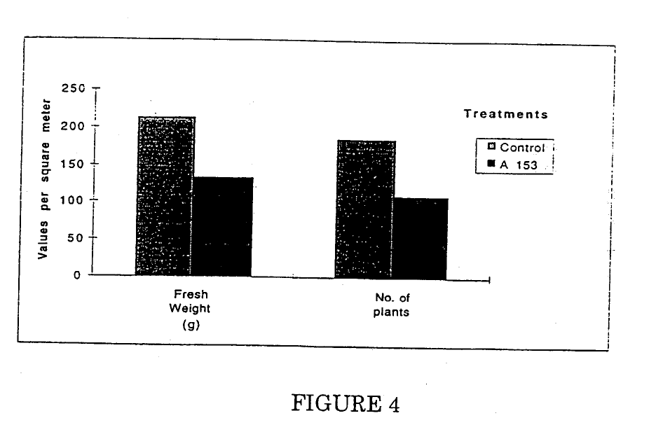

Treatment results were read in August, after the barley heading, by cutting off clover plants as well as various other sporadic weeds present at ground level and determining shoot biomass and number of plants, in three randomly chosen 0,25 square meter areas per plot. The cut-off clover and weeds were taken to the laboratory where both fresh and dry weights were recorded. Typical results from one of the experiments are shown in FIG. 4.

### EXAMPLE 17

### Effects on Field Pennycress (Thlaspi arvense) and Chickweed (Stellaria media) of Spraying A 153 Broth and A 153 Cell-free Supernatant in Two Microplot Field Experiments

The microplot field experiments, designed as randomized blocks with six repetitions, had plot sizes of about 800 cm² for Thlaspi arvense and 1 m² for Stellaria media, and were located in Uppsala on loamy soils with about 3 per cent humus content.

Bacterial suspensions for spraying was produced as described under Example 16 above, and the cell-free supernatant tested was produced by first centrifuging the bacterial culture twice for 10 minutes at 10000 g (Sorvall RC 5B) at 4° C., and then vacuum filtrate the resulting supernatant through a 0,45 μm Millipore system. The filtrates were then refrigerated or frozen for later use. The treated plots were sprayed at the late four-leaf stage using a conventional compression sprayer. Control plots were sprayed with tap water. Care was taken not to spray in strong sunlight and not directly in connection to rain, e. g preferably late in the afternoon on cloudy weather with no wind.

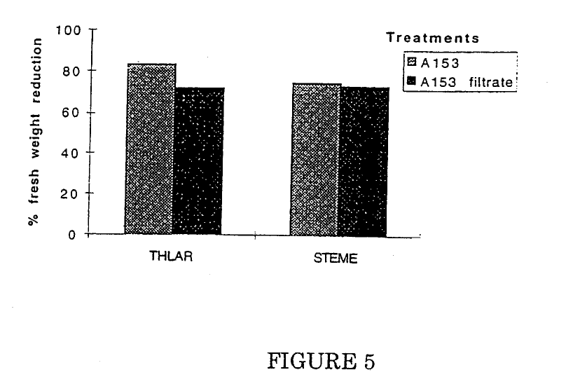

Treatment results were read about three weeks after spraying by cutting off and weighing the shoot of the weed plants in the whole microplot. The cut-off weeds were taken to the laboratory where both fresh and dry weights were recorded. Typical results from one of the experiments are shown in FIG. 5.

### EXAMPLE 18

### The Effect of Various Preparations of the S. plymuthica A153 Broth Culture and Haterumalide B on Growth of Stellaria media

Fifty per cent strength TSB medium (TSB, 15 g Tryptic Soy Broth (Difco Ltd) and 1000 ml deionized H₂O) were used to culture all bacterial isolates. The cultures, 50 ml, 250 ml and 1000 ml, respectively, were grown in 100 ml, 500 ml and 2000 ml Erlenmeyer flasks and were initiated either by transferring a single colony from the TSA plate or by transferring 0.1 ml of a 24-hours old starter inoculum per 5 ml of fresh medium. The cultures were incubated on a rotary shaker (120 rev min⁻¹) for 48 hours at 20° C. in darkness, and the cell densities obtained were then approximately from 1 to 1.5×10⁰ cfu ml⁻¹. Only 48-hours old cultures were used in the screening and purification procedures

Preparations of the culture of the isolate A 153, namely cell free supernatant, washed cells suspended respectively in buffer and in TSB, cell lysate as well as ethyl acetate and aqueous phase residue extracts of the cell free supernatant and purified haterumalide B, were tested for their effects on growth of Stellaria media, a typical plant weed.

Bacterial cell free supernatants were obtained by centrifugation (Sorvall RC 5B centrifuge, 20000×g, 20 min, 4° C. or 15000×g, 30 min, 4° C.) and subsequent filter-sterilization (Akafilter, Västra Frolunda, Sweden). Washed bacterial cells were prepared by collecting cell pellets after centrifugation, washing the cells once with deionized water, centrifuging after washing, and finally suspending the cells to the initial volume with either buffer or TSB. To obtain cell lysate, cells were lysed overnight with 4-times acetone volume of the initial culture volume, and acetone was then evaporated to dryness. The remaining material was finally redissolved in 50% MeOH/50% H₂O.

Further preparation was done on the cell free supernatant, which was continuously extracted, using 0.2 or 1.8 1 of supernatant, with 0.15 and 0.5 l, respectively, of recirculating ethyl acetate (EtOAc), for 18 hours at 105° C. The ethyl acetate extract (EtOAc extract) and the aqueous phase extract (AqPh. extract) were collected, and the resulting material was then evaporated to dryness. Dried EtOAc and AqPh. extracts were finally dissolved using minimal volumes of methanol (MeOH) and 1:1 MeOH/H₂O, respectively, and stored at −20° C. for following analysis.

All preparations were prepared in such a way that their relative concentrations corresponded to these of the bacterial culture, i.e. 0.75×10⁸ cfu ml⁻¹ (non diluted, 1×) and 7,5×10⁸ cfu ml⁻¹ (concentrated 10×), and their effect on Stellaria media was compared to the effect of bacterial culture with around 0.75×10⁸ cfu ml⁻¹. Three pots, with 4 Stellaria plants in each pot, were sprayed with 4 ml of tested preparations per treatment, and the controls were: Stellaria plants treated with 50% TSB and 50 and 10% methanol (MeOH) in deionized H₂O. The last-mentioned controls were included since the highest MeOH concentration in EtOAc extract and AqPh. extract preparations was around 50%.

The effect of various culture preparations on Stellaria are presented as a number of plants surviving the treatments and a shoot fresh weight of plants and are summarized in Table 9 below.

TABLE 9
Response of Stellaria media (6-8 leaf stage) to foliar
spraying with A 153 culture, supernatant, extracts and Haterumalide
B (4 ml/4 plants/pot; three replicate pots)

| Treatment | Plants Surviving | Shot Fresh Weight (g) |
|---|---|---|
| Control - TSB | 4.0 | 5.0 |
| Control - MeOH 10% | 4.0 | 4.1 |
| Control - MeOH 50% | 4.0 | 4.2 |
| Broth Culture | 1.7 | 0.5 |
| Supernatant | 0.3 | 0.1 |
| Cells in Buffer | 4.0 | 5.4 |
| Cells in TSB | 4.0 | 2.1 |
| Cell Lysate 1× | 4.0 | 3.5 |
| Cell Lysate 10× | 3.0 | 0.9 |
| EtOAc extract 1× | 4.0 | 4.6 |
| EtOAc extract 10× | 3.7 | 2.4 |
| AqPhase Extract 10× | 0.0 | 0.1 |
| Haterumalide B | 0.0 | 0.1 |

### EXAMPLE 19

### The Effect of Haterumalide B on Differentiation and Growth of Lemna minor and on Green Algae (Scenedesmus quadricauda)

Twenty four-well microtiter plates were used to test the effect of the purified haterumalide B on differentiation and growth of Lemna minor in a concentration range between 1 μg ml⁻¹ and 60 μg ml⁻¹. To obtain respective concentrations of the tested metabolite, the appropriate volume out of the respective metabolite stock solutions of 100 μg ml⁻¹ and 10 μg ml⁻¹, dissolved in acetonitrile, was distributed into the wells, and the solvent was then evaporated. Thereupon, the metabolite was dissolved in a sterile Lemna medium and three Lemna minor plants with 10 fronts were placed into each well. Tests were done in duplicate and controls were Lemna minor plants placed in only Lemna medium.

Haterumalide B totally inhibited the differentiation and growth of Lemna minor at all tested concentrations. Results are presented in Table 10 below.

TABLE 10

| | Number of Lemna minor fronts (lea- | | |
|---|---|---|---|
| Haterumalide B con- | ves) per one microtiter plate well | | |
| centration (μg ml-1) | Beginning of test | End of test | Visible effects |
| 60 | 10 | 10 | At all tested concentrations |
| 30 | 10 | 10 | of haterumalide B Lemna |
| 10 | 10 | 10 | fronts impaired and totally |
| | | | chlorotic |
| 5 | 10 | 10 | |
| 1 | 10 | 10 | Lemna fronts intact and in- |
| Control | 10 | 36 | tensively green |

Additionally, the similar method was used to preliminary test the effect of haterumalide B on the growth of green algae (Scenedesmus quadricauda). Evaporated haterumalide B was dissolved in a sterile Z8 medium with the suspension of algae. Tests were performed in duplicate with suspensions of Scenedesmus quadricauda placed in only sterile Z8 medium as controls. The preliminary results show that haterumalide B inhibits the growth of green algae at concentrations similar to those used for the Lemna minor test.

Haterumalides A, B, E and X and derivatives thereof are also contemplated for use as antibacterial and antiviral agents. Haterumalide X is further contemplated for use as a cytostatic agent.

### References

Adams, P. B. (1990). The potential of mycoparasites for biological control of plant diseases. Annu Rev Phytopathol 28, 59-72.

Dueck, J., Morrall, R. A. A., and McKenzie, D. L. (1983). Control of Sclerotinia sclerotiorum in rapeseed with fungicides. Can J Plant Pathol 5, 289-293.

Duijff, B. J., de Kogel, W. J., Bakker, P. A. H. M., and Schippers, B. (1994). In fluence of pseudobactin 358 on the iron nutrition of barley. Soil biology and biochemistry 26, 1681-1688.

Gerlagh, M., Goossen Van De Geijn, H. M., Fokkema, N. J., and Vereijken, P. F. G. (1999). Long-term biosanitation by application of Coniothyrium minitans on Sclerotinia sclerotiorum-infected crops. Phytopathology. 89, 141-147.

Harrison, L. A., Letendre, L., Kovacevich, P., Pierson, E., and D., W. (1993). Purification of an antibiotic effective against Gaeumannomyces graminis var tritici produced by a biocontrol agent, Pseudomonas aureofaciens. Soil biology and biochemistry 25, 215-221.

Howell, C. R., and Stipanovic, R. D. (1979). Control of Rhizoctonia solani on cotton seedlings with Pseudomonas fluorescens and an antibiotic produced by the bacterium. Phytopathology 69, 480-482.

Howell, C. R., and Stipanovic, R. D. (1980). Suppression of Pythium ultimum induced damping-off of cotton seedlings by Pseudomonas fluorescens and its antibiotic pyoluteorin. Phytopathology 70, 712-715.

Hökeberg, M. (1998). Seed bacterization for control of fungal seed-borne diseases in cereals. Doctoral, Plant Pathology and Biocontrol Unit, , , SLU, Uppsala.

Jacobi, M., Winkelmann, G., Kaiser, D., Kempler, C., Jung, G., Berg, G., and Bahl, H. (1996). Maltophilin: a new antifungal compound produced by Stenotrphomonas maltophilia R3089. J. Antibiot. 49, 1101-1104.

Kalbe, C., Marten, P., and Berg, G. (1996). Strains of the genus Serratia as beneficial rhizobacteria of oilseed rape with antifungal properties. Microbiological research, 433-439.

Kang, Y., Carlson, R., Tharpe, W., and Schell, M. A. (1998). Characterization of genes involved in the biosynthesis of a novel antibiotic from Burkholderia cepacia BC11 and their role in biological control of Rhizoctonia solani. Applied and evironmental microbiology 64, 3939-3947.

Kharbanda, P. D., and Tewari, J. P. (1996). Integrated management of canola diseases using cultural methods. Can J Plant Pathol 18, 168-175.

Kraus, J., and Loper, J. E. (1995). Characterization of a genomic region required for production of the antibiotic pyoluteorin by the biological control agent Pseudomonas fluorescence Pf-5. Appl. Environ. Microbiol. 61, 849-854.

Lambert, B., Leyns, F., van Rooyen, F., Gossele, F., Papon, Y., and Swings, J. (1987). Rhizobacteria of maize and their antifungal activities. Appl. Environ. Microbiol 53, 1866-1871.

Lee, C., Kim, S., Hyun, B., Suh, J., Yon, C., Kim, Y., Lim, C., and Kim, C. (1994). Cepacidine A, a novel antifungal antibiotic produced by Pseudomonas cepacia. J. Antibiot. 47, 1402-1418.

Lüth, P., Schulz, R. R., and Pfeffer, H. (1993). The influence of bacterial antagonists on the infestation of a soil as well as on the yield of winter oilseed rape affected by Sclerotinia sclerotiorum. Zentralbl Mikrobiol 148, 32-38.

Martin, F. N. (1992). Pythium. In “Methods for research on soilborne phytopathogenic fungi” (L. L. Singleton, J. D. Mihail and C. M. Rush, eds.). APS Press, St Paul, Minn.

Mc Lean, D. M. (1958). Some experiments concerned with the formation and inhibition of apothecia of Sclerotinia sclerotiorum (Lib.) de Bary. Plant Dis Rep 4, 409-412.

McLoughlin, T. J., Quinn, J. P., and R, B. (1991). Biological control of Sclerotinia sclerotiorum on sunflower by Pseudomonas cepacia. In “Second International Workshop on Plant Growth-Promoting Rhizobacteria” (C.

Keel, B. Koller and G. Defago, eds.), pp. 201. Bulletin of the IOBC WPRS (International Organization for Biological and Integrated Control of Noxious Animals and Plants, Interlaken, Switzerland.

McLoughlin, T. J., Quinn, J. P., Bettermann, A., and Bookland, R. (1992). Pseudomonas cepacia suppression of sunflower wilt fungus and role of antifungal compounds in controlling the disease. Appl. Environ. Microbiol. 58, 1760-1763.

McQuilken, M. P., Mitchell, S. J., Budge, S. P., Whipps, J. M., Fenlon, J. S., and Archer, S. A. (1995). Effect of Coniothyrium minitans on sclerotial survival and apothecial production of Sclerotinia sclerotiorum in field-grown oilseed rape. Plant Pathol 44, 883-896.

Meyers, E., G. S., Bisacchi, G. S., Dean, L., Liu, W. C., Minassian, B., Slusarchyk, D. S., Sykes, R. B., Tanaka, S. K., and Trejo, W. (1987). Xylocandin: a new complex of antifungal peptides. I. Taxonomy, isolation and biological activity. J. Antibiot. 40, 1515-1529.

Nakayama, T., Homma, Y., Hasidoko, Y., Mizutani, J., and Tahara, S. (1999). Possible role of xanthobaccins produced by Stenotrophomonas sp. strain SB-K88 in suppression of sugar beet damping-off disease. Appled and environmental microbiology 65, 4334-4339.

Okamoto, H., Sato, M., Sato, Z., and Isaka, M. (1998). Biocontrol of Phytophtora capsici by Serratia marcescens F-1-1 and analysis of biocontrol mechanisms using transposon-insertion mutants. Ann Phytopathol Soc. Jpn 64, 287-293.

Pozdnyakov, V. N. (1994). Soil bacteria-antagonists of phytopathogenic microflora in control of plant diseases. In “Third International Workshop on Plant Growth-Promoting Rhizobacteria” (V. N. Pozdnyakov, M. H. Ryder, P. M. Stephens and G. D. Bowen, eds.), pp. 34-35. CSIRO Australia, Adelaide, South Australia, Australia.

Raaijmakers, J. M., Weller, D. M., and Tomashow, L. S. (1997). Frequency of antibiotic producing Pseudomonas spp. in natural environments. Appl.

Environ. Microbiol. 63, 881-887.

Rhodes, D.J. (1993). Formulation of biological control agents. Exploitation of Microorganisms, edited by D.G. Jones, Chapman & Hall, London 1993, 411-439.

Shoji, J., Hinoo, H., Sakazaki, R., Kato, T., Hattori, T., Matsumoto, K., Tawara, K., Kikuchi, J., and Terui, Y. (1989). Isolation of CB.25-I, an antifungal antibiotic, from Serratia plymuthica. J. Antibiot. 42, 869-874.

Steadman, J. R. (1979). Control of plant diseases caused by Sclerotinia species.

Phytopathology, 904-907.

Steadman, J. R., and Nickerson, K. W. (1975). Differential inhibition of sclerotial germination in Whetzelinia sclerotiorum. Mycopathologia 57, 165-170.

Stierle, D. B., Stierle, A. A., and Ganser, B. K. (1999). Isolation of thwo highly methylated polyketide derivatives from a yew-associated Penicillium species. J. Nat. Prod. 62, 1147-1150.

Strobel, G., Li, J., Sugawara, F., Koshino, H., Harper, J., Hess, W. M., and Li, J. Y. (1999). Oocydin A, a chlorinated macrocyclic lactone with potent antioomycete activity from Serratia marcescens. Microbiology Reading 145, 3557-3564.

Takada, N., Sato, H., Suenaga, K., Arimoto, H., Yamada, K., Ueda, K., and Ue mura, D. (1999). Isolation and structures of haterumalides NA, NB, NC, ND, and NE, novel macrolides from an Okinawan sponge Ircinia sp. Tetrahedron Lett. 40, 6309-6312.

Teintze, M., Hossain, M. B., Baines, C. L., Leong, J., and D., v. d. H. (1981). Structure of ferric pseudobactin, a siderophore from a plant growth promoting Pseudomonas. Biochemistry 20, 6446-6457.

Thaning, C., and Nilsson, H. E. (2000). A narrow range of wavelengths active in regulating apothecia development in Sclerotinia sclerotiorum. Journal of Plant Pathology In press.

Tomashow, L. S., and Weller, D. M. (1998). Role of phenazine antibiotics from Pseudomonas fluorescens in biological control of Gaeumannomyces graminis var. tritici. J. Bacteriol. 170, 3499-3508.

Trutmann, P., Keane, P. J., and Merriman, P. R. (1982). Biological control of Sclerotinia sclerotiorum on aerial parts of plants by the hyperparasite Coniothyrium minitans. Trans Brit Mycol Soc 78, 521-529.

Turner, G. J., and Tribe, H. T. (1975). Preliminary field plot trials on control of Sclerotinia trifoliorum by Coniothyrium minitans. Plant Pathol 24, 109-113.

Turner, G. J., and Tribe, H. T. (1976). On Coniothyrium minitans and its parasitism of Sclerotinia species. Trans Brit Mycol Soc 66, 97-105.

Ueda, K., and Hu, Y. (1999). Haterumalide B: A new cytotoxic macrolide from an Okinawan Ascidian Lissoclinum sp. Tetrahedron Lett. 40, 6305-6308.

Whipps, J. M., and Budge, S. P. (1990). Screening for sclerotial mycoparasites of Sclerotinia sclerotiorum. Mycological Research 94, 607-612.

Yuen, G. Y., Godoy, G., Steadman, J. R., Kerr, E. D., and Craig, M. L. (1991). Epiphytic colonization of dry edible bean by bacteria antagonistic to Sclerotinia sclerotiorum and potential for biological control of white mold disease. Biological Control 1,293-301.

Åström, B., and Gerhardson, B. (1988). Differential reactions of wheat and pea genotypes to root inoculation with growth-affecting rhizosphere bacteria. Plant Soil 109, 263-269.

1. The Serratia plymuthica strain designated A135 and deposited at The National Collection of Industrial and Marine Bacteria, Aberdeen, Scotland and having received the NCIMB accession number 40938, and biologically pure cultures thereof.

2. A weeds controlling composition comprising, as active ingredient, the microorganism claimed in claim 1 or its culture broth or a fraction thereof, optionally in admixture with an agriculturally, silviculturally or horticulturally acceptable carrier or diluent and optionally additives conventionally used in such a composition.

3. A fungal growth controlling composition comprising, as active ingredient, the microorganism claimed in claim 1 or its culture broth or a fraction thereof, optionally in admixture with an agriculturally, silviculturally or horticulturally acceptable carrier or diluent and optionally additives conventionally used in such a composition.

4. The weeds controlling composition according to claim 2 where the weed to be controlled is an annual herbaceous weeds.

5. The weeds controlling composition according to claim 2 where the weed to be controlled is Stellaria media, Thlaspi arvense, Chenopodium album, Viola arvensis, or Capsella-bursa-pastoris.

6. The fungal growth controlling composition according to claim 3 where the fungus to be controlled is Aspergillus fumigatus, Fusarium culmorum, Fusarium oxysporum, Heterobasidion annosum, Microsporum cannis, Pythium ultimum or Sclerotinia sclerotiorum.

7. The compound haterumalide X of the formula

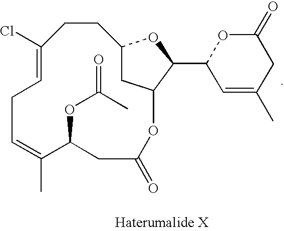

8. Use of the compounds haterumalide B, E och X of the formulae 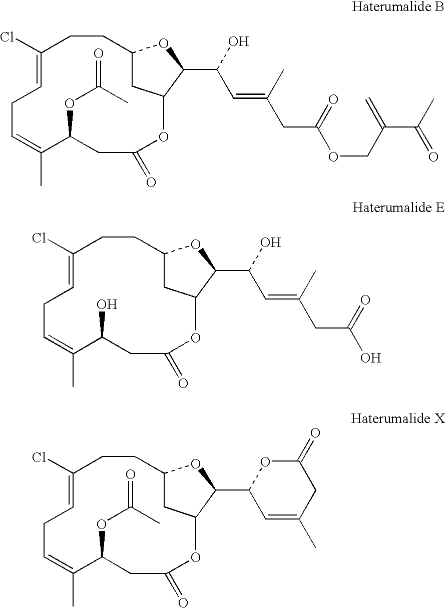
and derivatives thereof as biofungicides for controlling fungal diseases of plants, humans and animals.

9. The use according to claim 8 for controlling plant diseases caused by pathogenic fungi.

10. The use according to claim 8 or 9 for controlling diseases caused by pathogenic fungi in crops.

11. The use according to claim 8, 9 or 10 for controlling sclerotia-producing fungi.

12. The use according to claim 11 for controlling diseases caused by Sclerotinia sclerotiorum.

13. The use according to claim 11 or 12 for controlling fungal diseases in bean, soybean, sunflower and oil seed brassicae crops.

14. The use according to claim 8 for treatment of diseases of humans and animals caused by pathogenic fungi.

15. The use according to claim 14 for treatment of diseases caused by Aspergillus spp., Candida spp. and Fusarium spp.

16. Use of the compounds haterumalide A, B, E and X of the formulae 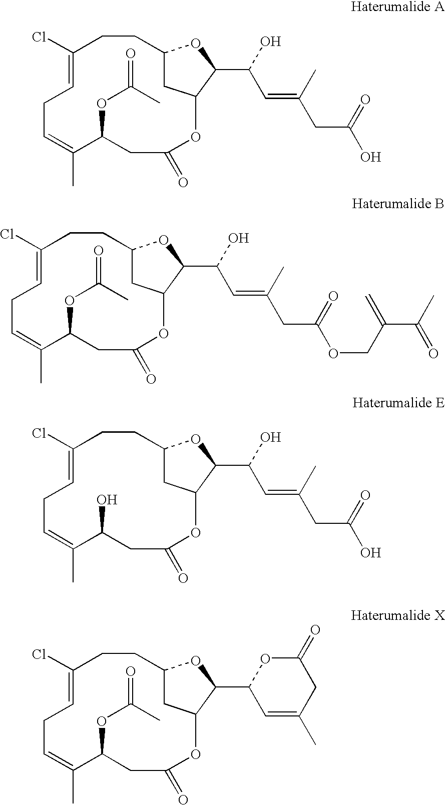
and derivatives thereof as bioherbicides for controlling weeds.

17. The use according to claim 16 for controlling fat hen (Chenopodium album), field pennycress (Thlaspi arvense), redroot pigweed (Amaranthus retroflexus L.), charlock (Sinapsis arvensis L.), field pansy (Viola arvensis), common chickweed (Stellaria media), Lemna minor, Fallopia convolvolus, Equisetum arvense, Galeopsis spp., Polygonum spp., and Fumaria officinalis.

18. The use according to any of claims 8-15 of haterumalide B, E och X and derivatives thereof contained in a fungal disease controlling composition.

19. The use according to claim 16 or claim 17 of haterumalide A, B, E and X and derivatives thereof contained in a weeds controlling composition.

20. Plant fungal disease controlling composition for use according to claim 18, characterized by comprising haterumalide B, E and/or X and/or derivatives thereof, optionally in admixture with an agriculturally, silviculturally or horticulturally acceptable carrier or diluent, and optionally conventional agricultural or horticultural additives.

21. Human or animal fungal disease controlling composition for use according to claim 18, characterized by comprising haterumalide B, E and/or X and/or derivatives thereof, optionally in admixture with a pharmaceutically or veterinarily acceptable carrier or diluent and optionally conventional pharmaceutical additives.

22. Weeds controlling composition for use according to claim 19, characterized by comprising haterumalide A, B, E and/or X and/or derivatives thereof, optionally in admixture with an agriculturally, silviculturally or horticulturally acceptable carrier or diluent, and optionally conventional agricultural or horticultural additives.

23. A process for the preparation of haterumalide A, B, E and X, characterized by cultivating, under conditions appropriate for obtaining the respective compound, a Serratia plymuthica strain and optionally isolating and purifying the respective haterumalide compound from the culture broth.

24. The process according to claim 23, characterized in that the Serratia plymuthica strain used is A 153 (NCIMB 40938).

25. A method of controlling plant diseases caused by pathogenic fungi by applying an antifungally effective dose of a biocontrol agent in the environment where the disease is to be controlled, characterized in that the biocontrol agent is the Serratia plymuthica strain A153 (NCIMB 40938) according to claim 1 or a composition according to claim 3 or claim 6.

26. A method of controlling weeds by applying an effective weeds controlling dose of a biocontrol agent in the environment where the weed is to be controlled, characterized in that the biocontrol agent is the Serratia plymuthica strain A153 (NCIMB 40938) according to claim 1 or a composition according to any of claims 2, 4 and 5.

27. A method of controlling plant diseases caused by pathogenic fungi by applying an antifungally effective dose of an active agent in the environment where the disease is to be controlled, characterized in that the active agent is haterumalide B, E and/or X and/or a derivative thereof or a composition according to claim 20.

28. A method of controlling weeds by applying an effective weeds controlling dose of an active agent in the environment where the weed is to be controlled, characterized in that the active substance is haterumalide A, B, E and/or X and/or a derivative thereof or a composition according to claim 22.

29. The method according to claim 25 or claim 27 for controlling fungal diseases of plants, characterized by applying the biocontrol agent or the active agent by seed, foliar, root, soil or growth substrate application.

30. The method according to claim 26 or claim 28 for controlling weeds, characterized by applying the biocontrol agent or the active agent by spraying on crops or soil.

31. The method according to claim 25 or claim 27, characterized by applying the biocontrol agent or the active agent to plant vegetative propagation units.

32. The method according to any of claims 25, 27, 29 or 31, characterized by applying the biocontrol agent or the active agent to bean, soybean, sunflower and brassicae crops to control Sclerotinia sclerotiorum.

33. The use according to claim 8 for providing fungicidal or fungistatic effects or effects regulating cell division, cell elongation and/or cell differentiation, the effects being induced by haterumalide A, B, E and X and derivatives thereof.

34. Use of haterumalide X and derivatives thereof as cytostatic agent.

35. Use of haterumalide A, B, E and X and derivatives thereof as antibacterial and antiviral agents.

36. Use of A153 and haterumalide A, B, E and X and derivatives thereof for controlling the growth of algae.
